(19) United States
(12) Patent Application Publication
(10) Pub. No.: US 2026/0183306 A1
(43) Pub. Date: Jul. 2, 2026

(54) Methods for Treating Respiratory Diseases Characterized by Mucus Hypersecretion

(71) Applicant: The Board of Trustees of the Leland Stanford Junior University, Stanford, CA (US)

(72) Inventors: Jeffrey Axelrod, Stanford, CA (US); Carlos Milla, Stanford, CA (US); Eszter Vladar, Stanford, CA (US)

(21) Appl. No.: 19/131,194

(22) PCT Filed: Feb. 15, 2023
(86) PCT No.: PCT/US2023/013111

§ 371 Date: May 19, 2025

Related U.S. Application Data
(60) Provisional application No. 63/427,634, filed on Nov. 23, 2022.

(51) Int. Cl. A61K 31/55; A61K 31/145; A61K 31/192; A61K 31/381; A61K 31/417; A61K 31/4245; A61K 31/5513; A61K 31/675; A61K 38/05; A61P 11/00

(52) U.S. Cl. CPC A61K 31/55; A61K 31/145; A61K 31/192; A61K 31/381; A61K 31/417; A61K 31/4245; A61K 31/5513; A61K 31/675; A61K 38/05; A61P 11/00

(57) ABSTRACT

The invention therefore provides methods of treating a respiratory disease characterized by mucus hyper-secretion comprising administering to a human patient in need of such treatment a gamma secretase inhibitor (GSI), wherein the administration of a GSI is effective in reducing mucus in such patient's lungs or inhibiting mucus accumulation in said patient's lungs. In some embodiments, the methods of the invention are effective in treating a respiratory disease selected from the group consisting of cystic fibrosis, chronic obstructive pulmonary disease, primary ciliary dyskinesis, chronic bronchitis, asthma, idiopathic and secondary bronchiectasis, bronchiolitis obliterans, idiopathic pulmonary fibrosis, respiratory infection and mucus accumulation in response to infection. Methods of the invention further include methods of treating cystic fibrosis wherein a GSI is administered to a patient being administered or in need of a CFTR modulator, wherein the mucus in such patient's lungs is reduced or mucus accumulation in such patient's lungs is inhibited.

### CROSS-REFERENCE TO RELATED APPLICATIONS

Pursuant to 35 U.S.C. § 119 (e), this application claims priority to the filing date of U.S. Provisional Patent Application Ser. No. 63/427,634 filed Nov. 23, 2022, the disclosure of which application is incorporated herein by reference in its entirety.

### ACKNOWLEDGEMENT OF GOVERNMENT RIGHTS

This invention was made with Government support under contract GM098582 awarded by the National Institutes of Health. The Government has certain rights in the invention.

### BACKGROUND

Airway epithelial cells include a mixture of predominantly multiciliated cells (MCCs) and mucus-secreting goblet cells exposed at the luminal surface and underlying basal (stem) cells. MCCs each possess 200 to 300 motile cilia that beat in a coordinated, directional manner to propel inhaled contaminants trapped by the mucus layer out of the lungs. (Tilley A E, Walters M S. Shaykhiev R, Crystal R G. Cilia dysfunction in lung disease. Annu Rev Physiol. 2015; 77:379-406). Goblet cells secrete mucus that forms a protective barrier for the respiratory epithelia, and they can increase in activity and number in response to noxious stimuli such as infection. Breakdown of airway clearance can precipitate and/or exacerbate acute infections and chronic inflammatory conditions such as cystic fibrosis (CF), primary ciliary dyskinesia (PCD), chronic rhinosinusitis (CRS), chronic obstructive pulmonary disease (COPD), and asthma (Id.).

CF is regarded as the most severe mucociliary clearance disorder. (Bruscia E M. Bonfield T L. Innate and adaptive immunity in cystic fibrosis. Clin Chest Med. 2016; 37(1):17-29). Mutations in the CF transmembrane conductance regulator (CFTR) lead to dehydration of the mucosal surface and accumulation of thick, abnormal mucus that both hinders airway clearance and serves as a site for polymicrobial infections. These events contribute to severe, chronic inflammation and to cycles of repeated injury and imperfect repair. These in turn bring about epithelial dysfunction, which includes structural and functional changes such as hyperplasia of mucus-secreting cells, decrement in MCC numbers, abnormal tissue architecture with scarring, diminished barrier function, and decreased regenerative capacity. (Adam D, et al. Cystic fibrosis airway epithelium remodelling: involvement of inflammation. J Pathol. 2015; 235(3):408-419). CF patients march down an inevitable slope of airway destruction in the form of bronchiectasis, chronic cough, dyspnea, sinusitis, recalcitrant infection with recurrent antibiotic use, and oxygen dependence. Epithelial dysfunction in CF is thought to be a major factor in disease progression, ultimately resulting in lung transplantation once medical options become exhausted. (Regamey N. Jeffery P K, Alton E W, Bush A, Davies J C. Airway remodeling and its relationship to inflammation in cystic fibrosis. Thorax. 2011; 66(7):624 629).

A functional balance of secretory cell derived mucus secretion and MCC driven motility results in an effective mucociliary clearance process that is essential for respiratory health. MCCs are terminally differentiated and arise from the basal cells or secretory cell types of the airway epithelium beginning in embryonic development and continuing as a regenerative process throughout life. (Hogan B L, et al. Repair and regeneration of the respiratory system: complexity, plasticity, and mechanisms of lung stem cell function. Cell Stem Cell. 2014; 15(2):123-138; Rock J R, et al. Basal cells as stem cells of the mouse trachea and human airway epithelium. Proc Natl Acad Sci USA. 2009; 106(31):12771-12775). MCC differentiation starts with a Notch signaling event, in which cells respond to activation of the Notch transmembrane protein to become secretory cells, whereas ligand-expressing cells not responsive to Notch are directed to the MCC fate via an MCC-specific gene expression program that drives differentiation and ultimately the production of hundreds of regulatory and structural components required for motile cilium biogenesis. (Choksi S P, Lauter G, Swoboda P. Roy S. Switching on cilia: transcriptional networks regulating ciliogenesis. Development. 2014; 141(7):1427-1441). Robust mucociliary clearance requires production of cilia of the correct number, length, beat frequency and waveform, and, importantly, correct directionality along the tissue axis. Furthermore, inhibition of Notch signaling in differentiated epithelia has also been shown to shift cellular composition away from secretory and toward MCC cell fate by inducing transdifferentiation of secretory cells into MCCs (Lafkas et al. Nature 2015 Dec. 3; 528(7580):127-31).

Airway epithelia from patients with CF and other chronic inflammatory diseases have been shown to have sparse or absent MCCs, defective mucociliary clearance, and related decreased barrier function and regenerative capacity. In vitro and animal models have shown that by suppression of Notch signaling, gamma secretase inhibitors are able to restore a healthy balance of secretory and MCC cells both by driving de nova MCC differentiation and by promoting transdifferentiation of mature secretory cells into MCCs, thereby rescuing these cellular composition, barrier and regenerative phenotypes. (Vladar E K, Nayak J V, Milla C E, Axelrod J D. Airway epithelial homeostasis and planar cell polarity signaling depend on multiciliated cell differentiation. JCI Insight. 2016; 1(13); e88027). Further, transdifferentiation of mature secretory cells by gamma secretase inhibitors is relatively rapid, as compared to new cell differentiation, which is relatively slow.

Recent advances in the treatment of cystic fibrosis have led to the development of a class of drugs known as CFTR modulators. These drugs are an example of personalized medicine in that they are designed to treat individuals carrying specific CFTR mutations. CFTR modulators can be classed into three main classes: potentiators, correctors and premature stop codon suppressors, or read-through agents. CFTR potentiators increase the open probability of CFTR channels that have gating or conductance mutations. CFTR correctors are designed to increase the amount of functional CFTR protein delivered to the cell surface. CFTR read-through agents are designed to “force” read-through of premature stop codons, leading to the production of more full-length CFTR protein. (Derichs, N., Eur. Resp. Rev., 2013: 22: 127, 58-65). CFTR amplifiers are a type of CFTR modulator being developed and tested, and are designed to increase the amount of CFTR protein a cell makes at the transcriptional level, thereby potentially enhancing function in patients with CFTR mutations that lead to insufficient protein at the cell surface.

While CFTR modulators improve CFTR function in patients having the corresponding CFTR mutations, the modulators do not affect the altered cellular composition, damage to epithelial cell architecture and corresponding epithelial dysfunction. Improved therapies are needed for restoring MCC function and improving mucociliary clearance in cystic fibrosis and other diseases characterized by mucus hypersecretion and/or inadequate mucociliary clearance.

Gamma secretase inhibitors (GSIs) have been widely studied as pharmacologic agents in the treatment Alzheimer's disease due to the role of gamma secretase in the formation of amyloid beta and plaque formation. (Bailen D M. Meredith J E. Zaczek R, Houston J G, Albright C F: (Jamma-secretase inhibitors for Alzheimer's disease: balancing efficacy and toxicity. Drugs R D. 2006.7: 87-97. Evin G, Sernee M F, Masters C L: Inhibition of gamma-secretase as a therapeutic intervention for Alzheimer's disease: prospects, limitations and strategies. CNS Drugs. 2006, 20: 351-372). In addition, the role of Notch signaling in human cancers has led to investigation of GSLs as potential therapies for various tumor types. (Shih I and Wang T, Notch Signaling. Gamma-Secretase Inhibitors. and Cancer Therapy, Cancer Res 2007.67(5); 1879-1882). The ability of GSIs to block Notch signaling has also led to proposals for use of GSIs in treating respiratory diseases association with epithelial cell dysfunction. (EP 2932966 A1). Gamma secretase is a multi-unit transmembrane protease complex, consisting of four individual proteins. It is an aspartyl protease that cleaves its substrates within the transmembrane region in a process called regulated-intramembrane-proteolysis (RIP). (Kroft, AF, Marton, R, and Porte, A, Recent Advances in the Identification of gamma Secretase Inhibitors To Clinically Test the Ab Oligomer Hypothesis of Alzheimer's Disease. J. Med. Chem 2009, 52:6169-6188). While gamma secretase has been of interest as a therapeutic target for several years, due to its complexity, obtaining a detailed understanding of its structure and an understanding of structure activity relationships has been challenging. Nevertheless, significant progress has been made in elucidating certain structure activity relationships. (See Wolfe. MS. Gamma-Secretase Inhibition and Modulation for Alzheimer's Disease, Curr Alzheimer Res. 2008; 5(2): 158-164).

GSIs can be classified into three general types based on where they bind to gamma secretase: (1) active-site binding GSIs, (2) substrate docking-site-binding GSIs, and (3) alternate binding site GSIs. The latter category can be further subdivided into carboxamide- and arylsufonamide-containing GSIs. (Kreft et al, at 6171).

Alzheimer's disease clinical trials have revealed toxicities believed to be associated with gamma secretase, inhibition. (David H. Henley. Karen L. Sundell. Gopalan Sethuraman. Sheric A. Dowsett Patrick C. May (2014) Safety profile of semagacestat, a gamma-secretase inhibitor: IDENTITY trial findings, Current Medical Research and Opinion, 30:10, 2021-2032).

Additional GSIs have been investigated for potential cancer therapeutics, and generally exhibit toxicities at high doses.

### SUMMARY OF THE INVENTION

It has now been surprisingly found that a low dose of gamma secretase inhibitors ((GSIs), is effective in reverting the cellular abnormalities seen in association with respiratory diseases characterized by mucus hypersecretion, and is effective at doses allowing therapeutic activity and expected to avoid or minimize the adverse effects previously associated with this class of molecules. It has further been found that GSIs administered in combination with a CFTR modulator is effective in correcting epithelial cell dysfunction in cystic fibrosis cell-based model systems (primary cells from patients), in contrast to certain prevailing concepts, and indeed the combination may be synergistic in improving CFTR ion channel function and epithelial cell correction.

The invention therefore provides methods of treating a respiratory disease characterized by mucus hyper-secretion comprising administering to a human patient in need of such treatment a GSI, wherein the administration of low dose GSI is effective in reducing mucus in such patient's lungs or inhibiting mucus accumulation in said patient's lungs. In some embodiments, the methods of the invention are effective in treating a respiratory disease selected from the group consisting of cystic fibrosis, chronic obstructive pulmonary disease, primary ciliary dyskinesis, chronic bronchitis, asthma, idiopathic and secondary bronchiectasis, bronchiolitis obliterans, idiopathic pulmonary fibrosis and other fibrotic lung disorders, respiratory infection including exacerbations in chronic respiratory disorders, and mucus accumulation in response to acute infection.

In some embodiments, the GSI is selected from the group consisting of semagacestat, avagacestat, GS-1, DBZ, L-685.458, BMS-906024, crenigascestat, MRK 560, nirogacestat, RO-4929097, MK-0752, itanapraced, LY-3056480, fosciclopirox, tarenflurbil, and begacestat.

In some embodiments, the GSI is selected from the group consisting of semagacestat, nirogacestat, MK-0752, RO-492907, or crenigacestat. In some embodiments, the GSI is a carboxamide based GSI.

In some embodiments, methods are provided for treating respiratory diseases characterized by mucus hypersecretion comprising systemically administering semagacestat in an amount of from about 0.1 mg to about 50 mg daily, wherein the administration of semagacestat is effective in reducing mucus in such patient's lungs or inhibiting mucus accumulation in such patient's lungs. In some embodiments, semagacestat is administered in an amount of from about 0.5 mg to about 40 mg daily. In some embodiments, semagacestat is administered in an amount of from about 0.5 mg to about 30 mg daily, or from about 0.5 mg to about 20 mg daily, or from about 0.5 mg to about 10 mg daily. For example, semagacestat may be administered in about 0.1 mg, 0.25 mg, 0.5 mg, 1 mg. 2.5 mg, 5 mg, 10 mg, 15 mg, 20 mg, 25 mg, 30 mg, 35 mg, 40 mg, 45 mg or 50 mg daily. Preferably, semagacestat is administered orally.

In an embodiment of the invention, a method is provided for treating a respiratory disease characterized by mucus hyper-secretion comprising systemically administering to a human patient in need of such treatment a therapeutically effective amount of semagacestat, wherein said patient's semagacestat plasma concentration at steady state following multiple dose administration comprises an AUC (area under the curve) less than 2100 ng·hr/mL, such as less than 1220 ng·hr/mL, wherein the systemic administration of semagacestat is effective in reducing mucus in such patient's lungs or preventing mucus accumulation in such patient's lungs. In some embodiments, upon multiple dose administration, said patient's steady state semagacestat plasma concentration comprises an AUC less than 1500 ng·hr/mL, less than 1200 ng·hr/mL, or less than 900 ng·hr/mL, such as an AUC less than 1220 ng·hr/mL, less than 600 ng·hr/mL, or less than 250 ng·hr/mL.

In further embodiments of the invention, methods are provided for treating cystic fibrosis comprising administering an effective amount of a GSI to a human patient taking a CFTR modulator, wherein the mucus in such patient's lungs is reduced or mucus accumulation in such patient's lungs is inhibited. In some embodiments, the GSI is selected from the group consisting of semagacestat, nirogacestat, MK-0752, RO-492907, or crenigacestat. In some embodiments, the GSI is semagacestat.

In certain of these embodiments, the CFTR modulator is selected from the group consisting of a CFTR potentiator, a CFTR corrector, a CFTR premature stop codon inhibitor, a CFTR amplifier and combinations thereof. In some embodiments, the CFTR modulator is selected from the group consisting of ivacaftor, lumacaftor, tezacaftor, elexacaftor and combinations thereof.

### BRIEF DESCRIPTION OF THE DRAWINGS

FIG. 1 shows dose response data of semagacestat in primary human nasal epithelial cells (HNECs) compared to untreated cells and DAPT positive control. MCCs are labeled in green (acetylated tubulin) and cell junctions are labeled in red (ECAD). The percentage of MCCs increases from its baseline at 15.625 nM semagacestat to its maximum at around 125 nM. Toxicity is observed at micromolar doses.

FIG. 2 shows the ratio of MCCs to total luminal cells in HNECs treated with DAPT and various doses of semagacestat.

FIG. 3 shows method of scoring the ratio of MCCs to non-ciliated cells in airway epithelia in mice treated systemically in vivo by intraperitoneal (IP) dosing of semagacestat. Airways from lung sections of similar size were labeled for all nuclei (red; DAPI) and MCC cell fate (green; FoxJ1 and blue; acetylated tubulin). Cells were scored and the percentage of MCCs determined prior to unblinding treatment condition. Otherwise wildtype mice carried FoxJ1::GFP to facilitate scoring of MCCs.

FIG. 4 shows the body weight at days 9, 24 and 30 with daily systemic (IP) administration of semagacestat and vehicle control.

FIG. 5 shows the ratio of MCCs to total cells at day 7 following a three-day treatment with DAPT and low and high doses of semagacestat, with vehicle control.

FIG. 6 shows the ratio of MCCs to total cells at day 31 following a three-week treatment with semagacestat, with vehicle control.

FIG. 7 shows the effect of GSI treatment during proliferation (prior to differentiation) and during differentiation of HNECs.

FIG. 8 shows the quantitation of MCCs per total luminal cells of the data of FIG. 7.

FIG. 9 shows the effects of GSI treatment duration on mature (ALI+30d) HNECs, treated with DAPT and semagacestat for one (ALI+30 to +37d) or two weeks (ALI+30 to +44d).

FIG. 10 shows the quantitation of MCCs per total luminal cells of the data of FIG. 9.

FIG. 11 shows the results of HNECs treated with DAPT and semagacestat during differentiation only (ALI+0 to +21d) from either the apical or basal surface.

FIG. 12 shows the quantitation of MCCs per total luminal cells of the data of FIG. 11.

FIG. 13 shows the effect of semagacestat and DAPT treatment in an epithelial culture model of chronic airway inflammation. HNEC cultures were treated with IL-13 from ALI+7 to 14 to induce inflammation. DAPT and semagacestat increase the percentage of MCCs in controls (left). IL-13 treatment increases the percentage of mucin positive secretory cells and decreases the percentage of MCCs. Subsequent DAPT and semagacestat treatment rescues cell composition, increasing the percentage of MCCs and decreasing the percentage of mucin positive secretory cells.

FIG. 14 shows the quantitation of MCCs per total luminal cells of the data of FIG. 13.

FIG. 15 shows representative Ussing chamber tracings of cultures treated with ETI, semagacestat, or both.

FIG. 16 shows representative tracings of Ussing-chamber short circuit currents (Isc) following treatment with semagacestat in wild-type and CF cells.

FIG. 17 shows Ussing-chamber Isc responses following treatment with semagacestat in wild-type and CF cells. Two wild-type control samples (WT) and two CF patient samples (CF 1; a rare allelic combination and CF2; a F509Δ homozygote) were studied. CFTR current activity were assessed by CFTR inhibitor response and were found to be as great as or greater than vehicle control currents in both wild-type controls and in CF samples. Values are normalized to the baseline current.

FIG. 18 shows that under in vitro treatment in combination with the CFTR modulator lumacaftor, semagacestat decreased mucus production in human CF samples with different CFTR mutations.

FIG. 19 shows the effect of treatment with semagacestat, lumacaftor and combinations on human CF samples with different CFTR mutations. Semagacestat is effective at increasing the percentage of MCCs in the presence of Lumacaftor, and the combination may be more effective for some donors than when either is applied alone.

FIG. 20 shows the quantitation of MCCs per total luminal cell data for healthy patient and CF donor 1 of FIG. 19.

FIG. 21 shows the effects on primary healthy and cystic fibrosis airway epithelial cells treated with semagacestat (LY45139) during differentiation only (ALI+0 to +21d).

FIG. 22 shows SEM of healthy and CF primary human airway epithelial cultures, showing that multiciliated cells formed under DAPT treatment are indistinguishable from those in untreated healthy cultures.

FIG. 23 shows the result of DAFT treatment in mature cystic fibrosis HNEC cultures, demonstrating that GSI treatment induces the formation of additional multiciliated cells in mature cystic fibrosis cultures, while untreated cultures do not differentiate any more multiciliated cells.

FIG. 24 shows the results of HNECs treated during differentiation (ALI+0 to +21d) with DAFT and high and low concentrations of the GSIs LY45139, PF-03084014, RO-4929097 and MK-0752.

FIG. 25 shows the quantitation of MCCs per total luminal cells of the data shown in FIG. 24.

FIG. 26 shows measurements of CFTR short-circuit current activity measured in Ussing chambers in cultures from CF patients treated with LY45139, Elexcaftor/Tezacaftor/Ivacaftor (or “ETI”), or both.

FIG. 27 shows measurements of CFTR current activity measured in Ussing chambers in cultures from CF patients treated with MK04752, ETI, or both.

FIG. 28 shows measurements of CFTR current activity measured in Ussing chambers in cultures from CF patients treated with MK04752, ETI, or combinations with reduced doses of Elexacaftor (E component of the ETI modulator combination).

FIG. 29 shows the results of the GSI DAFT treatment on ionocyte formation in HNECs treated with DAPT during differentiation only (ALI+0 to +21d).

FIG. 30 shows the effect of varying concentrations of GSI MK-0752 and Elexacaftor of ciliary beat frequency (CBF).

FIG. 31 shows ciliary beat frequency (CBF) and cilium length of HNEs treated with DAFT versus urn eased controls.

FIG. 32 shows the airway surface liquid (ASL) reabsorption characteristic of CF HNEC cultures treated with ETI, semagacestat, or both.

FIG. 33 shows images taken from a high-speed video recorded microscopic images of latex bead movement as a reflection of mucus transport by the ciliated surface of cell cultures. Cultures were of HNECs from two CF donors (F508del homozygotes) under treatment with vehicle control, ETI, Semagacestat (LY) or both treatments combined.

FIG. 34 shows the calculated bead movement of the cultures shown in FIG. 33.

FIG. 35 GSI treatment induces multiciliated cell formation. A. Primary human airway epithelial cells were treated during differentiation (ALI+0 to +21d) with a range of concentrations of LY450139 and labeled at ALI+21d with anti-acetylated α-Tubulin (green) and ECAD (red) antibodies to mark cilia and epithelial junctions. High concentration (10 μM) of LY450139 disrupted the epithelial junctions, but lower concentrations lead to a dose dependent increase in multiciliated cell numbers similar to DAPT. Scale bar, 50 μm. B. Quantitation of data shown in A. One-way ANOVA, ns=not significant, *=p-val<0.01, **=p-val<0.001.

FIG. 36 GSI treatment induces multiciliated cell formation in differentiating and mature airway epithelia. A. Primary human airway epithelial cells were treated with DAPT and LY450139 during pre-ALI only (ALI-5 to −1d=proliferation), during ALI only (ALI+0 to +21d=differentiation) or continuously during pre-ALI and ALI (ALI-5d to +21d=all times) and labeled at ALI+21d with anti-acetylated α-Tubulin (green) and ECAD (red) antibodies. Only GSI treatment during differentiation and all times increased multiciliated cell numbers. GSI treatment during pre-ALI (proliferation only and all times treatments) had no detrimental effect on multiciliated cells or overall epithelial structure. Scale bar, 50 μm. B. Quantitation of data shown in A. One-way ANOVA, ns=not significant, *=p-val<0.0001. C. Mature (ALI+30d) primary human airway epithelial cells were treated with DAPT and LY450139 for one (ALI+30 to +37d) or two weeks (ALI+30 to +44d), then labeled with anti-acetylated α-Tubulin (green) and ECAD (red) antibodies. GSI treatment induces the formation of additional multiciliated cells in mature cultures, while untreated cultures do not form any more multiciliated cells. Scale bar, 50 μm. D. Quantitation of data shown in C. One-way ANOVA, *=p-val<0.0001. E. Primary human airway epithelial cells were treated with DAFT and LY450139 during differentiation only (ALI+0 to +21d) from either the apical or basal surface, then labeled at ALI+21d with anti-acetylated α-Tubulin (green) and ECAD (red) antibodies. GSI treatment induces multiciliated cell formation via both apical and basal application. Apical treatment abrogates the air-liquid interface, which results in the poor epithelial structure and multiciliated cell differentiation in the untreated cultures, which is partially rescued by GSI treatment. Scale bar, 50 μm. F. Quantitation of data shown in E. Oneway ANOVA, *=p-val<0.01, **=p-val<0.001.

FIG. 37 GSI treatment induces multiciliated cell formation in vivo. A. Adult Foxj1-EGFP mice were treated with 10 mg/kg DAFT, 0.1 or 1 mg/kg LY450139 or vehicle control twice daily for three days, then on day 7 multiciliated cell number was quantitated in airway cross sections based GFP fluorescence, which shows that 1 mg/kg LY450139 increased multiciliated cell number. Treatment with 0.1 mg/kg LY450139 showed a similar trend but did not reach significance (p-val=0.09). n=4 mice were treated per category. Scale bar, 100 μm. B. Quantitation of data shown in A. One-way ANOVA, ns=not significant, *=p-val<0.05. C. Adult Foxj1-EGFP mice were treated with 1 mg/kg LY450139 or vehicle control once daily for 5 consecutive days for three weeks, then on day 30 multiciliated cell number was quantitated, which shows that 1 mg/kg LY450139 increased multiciliated cell number. n=8 mice were treated per category. Scale bar, 100 μm. D. Quantitation of data shown in C. T-test, *=p-val<0.01.

FIG. 38 GSI treatment induces multiciliated cell formation in epithelia with mucous cell hyperplasia. A. Primary human airway epithelial cells were treated with recombinant human IL-13 during early epithelial differentiation (ALI+7 to +14d), which induced mucous cell formation at the expense of multiciliated cell differentiation as assessed by anti-acetylated α-Tubulin (green), MUC5AC (red) and ECAD (blue) antibodies. These cultures were then treated with DAFT and LY450139 for an additional week (ALI+14 to +21d), then assessed at ALI+21d. While the untreated cells maintained the remodeled phenotype, GSI treatment induced multiciliated cell formation to levels comparable to healthy epithelia. Arrows point to acetylated α-Tubulin and MUCSAC double positive cells in GSI treated cultures. Scale bar, 50 μm. B. Quantitation of data shown in A. One-way ANOVA, *=p-val<0.0001.

FIG. 39 GSI treatment induces multiciliated cell formation in cystic fibrosis epithelia. A. Primary CF airway epithelial cells were cultured in with LY450139 with and without the CFTR modulators VX-445 and VX-659 during differentiation only (ALI+0 to +21d) and labeled at ALI+21d with anti-acetylated α-Tubulin (green) and ECAD (red) antibodies. Scale bar, 50 μm. B. The same cells were subjected to Ussing chamber short circuit current (Isc) measurements to measure CFTR activity following standardized stimulation protocols. Two-way ANOVA, *=p-val<0.0001.

FIG. 40 Multiciliated cell formation is induced by a variety of clinically tested GSIs. A. Primary human airway epithelial cells were treated during differentiation (ALI+0 to +21d) with DAPT and high and low concentrations of LY450139, PF-03084014, RO-4929097 and MK-0752 and labeled at ALI+21d with anti-acetylated α-Tubulin (green) and ECAD (red) antibodies to mark cilia and epithelial junctions. At high concentrations, GSIs disrupted epithelial structure, but at the low concentration all induced multiciliated cell formation, similar to DAPT. Scale bar, 50 μm. B. Quantitation of data shown in A. One-way ANOVA, ns=not significant. *=p-val<0.01, **=p-val<0.001 C. Multiciliated cells differentiated in the presence of DAPT treatment had a modest, but significant increase in ciliary beat frequency (t-test, p-val<0.0001), but showed no difference in cilium length (t-test, p-val=0.3111).

FIG. 41 GSI treatment induces structurally normal cilia in healthy and CF epithelia. SEM of healthy and CF primary human airway epithelial cultures shows that multiciliated cells formed under DAPT treatment are indistinguishable from those in untreated healthy cultures.

FIG. 42 GSI treatment and multiciliated cell quantitation in mouse airways. A. Schematic of GSI treatment timelines in mice. B. Mouse lung sections were immunolabeled with anti-GFP (green) and ac. α-Tubulin (blue) antibodies to identify multiciliated cells and stained with DAPI to identify nuclei. Nuclei without overlapping cytoplasmic GFP signal (white arrowhead and dots) were counted as nonciliated cells. Nuclei with overlapping cytoplasmic GFP signal (yellow arrowhead and dots) were counted as multiciliated cells. Ac. α-Tubulin labeling of cilia over the cell surface was used to provide additional confirmation of multiciliated cell identity, although ciliary axonemes are not always captured in the same section as the cell body. Images were quantitated by blinded visual scoring by three individuals. Scale bar, 100 μm. C. Mouse weights during Experiment 2.

FIG. 43 Impact of GSI treatment on CF HNEC differentiation and CFTR function. A. Impact of ALI culture medium formulation on multiciliated cell differentiation. ALIs from two CF donors were cultured with homemade (left, top) or commercial Pneumacult (right) medium and labeled at ALI+21d with anti-acetylated α-Tubulin (green) and ECAD (red) antibodies to mark cilia and epithelial junctions. LY450139 treatment in homemade media increases multiciliated cell number in CF cultures (left, bottom). Scale bar, 50 μm. B. Mature (ALI+30d) primary CF human airway epithelial cells were treated with DAPT for one (ALI+30 to +37d), then labeled with anti-acetylated α-Tubulin (green) and ECAD (red) antibodies. GSI treatment induces the formation of additional multiciliated cells in mature CF cultures, while untreated cultures do not differentiate any more multiciliated cells. Scale bar, 50 μm. C. Representative Using chamber results from one CF donor. D. Primary healthy airway epithelial cells were treated with DAPT during differentiation only (ALI+0 to +21d) and labeled at ALI+21d with anti-FOX (green), acetylated α-Tubulin (red) antibodies and stained with DAPI (blue) to mark nuclei. Both untreated and DAPT treated cultures contained a similar small number of FOXI1 positive nuclei indicative of ionocytes. Arrows point to rare FOXI1 positive ionocyte nuclei. Scale bar, 50 μm.

### DEFINITIONS

As used herein, a “disease characterized by mucus hypersecretion” means a disease wherein at least one pathology of the disease is due to presence of mucus at an epithelial surface in excess of the amount present under normal conditions. Included are diseases in which excess mucus is located in small airway passageways in which it is not normally present, and may be due to excess goblet cell production, hypertrophy of mucus glands, decreased MCCs. or other inadequate mucociliary clearance.

As used herein, an “effective amount” or “therapeutically effective amount” refers to an amount of the compound of the present disclosure that is effective to achieve a desired therapeutic result such as, for example decreasing goblet cell production and/or increasing production of multiciliated cells, thereby improving mucociliary clearance. In the context of the present invention, a desired therapeutic result includes reducing mucus production in such patient's lungs or inhibiting mucus accumulation in such patient's lungs. While the doses mentioned in the present disclosure are guidelines, an attending physician may adjust the dose according to the specific needs of the patient, including for example, severity of the disease, size and physical condition.

“Gamma secretase inhibitor(s)” or “GSI(s)” means a molecule capable of inhibiting or modulating the gamma secretase enzyme, and thereby inhibiting Notch signaling. Examples include DAPT (N-[(3,5-Difluorophenyl)acetyl]-L-alanyl-2-phenyl]glycine-1,1-dimethylethyl ester), semagacestat, avagacestat ((2R)-2-[[(4-Chlorophenyl)sulfonyl][[2-fluoro-4-(1,2,4-oxadiazol-3-yl)phenyl]methyl]amino]-5.5,5-trifluoropentanamide) (commercially available from www.tocris.com), DBZ (N-[(1S)-2-[[(7S)-6.7-Dihydro-5-methyl-6-ioxo-5H-dibenz[b.d]azcpin-7-yl anuno]−0.1-methyl-2-oxoethyl]-3.5-difluorobenzcneacetamide) (commercially available from www.tocris.com). L-685.458 ((5S)-(tert-Butoxycathonylanuno)-6-pbenyl-(4R)-hvdmxy-(2R)-benzvlbcxanovl l-L-leucy-L-phenylalaninamide) (commercially available from www.tocris.com), GS-1 (aka L-685458) (CAS Registry number 292632-98-5; WO0177144); BMS-906024 (bis(fluoroalkyl)-1,4-benzodiazepinone; CAS Registry Number 1401066-79-2), Crenigascestat (aka LY3039478) (Massard et al., “First-in-human study of LY3039478, a Notch signaling inhibitor in advanced or metastatic cancer,” J Clin Oncol (2015) 33(15_suppl): 2533), MRK 560 (N-[cis-4-[(4-Chlorophenyl)sulfonyl]-4-(2,5-difluorophenyl) yclohexyl]-1,1,1-trifluorotnethanesulfonamide) (commercially available from www.tocris.com), nirogacestat (aka PF-03084014)((S)-2-((S)-5,7-difluoro-1,2,3,4-tetrahydronaphthalen-3-ylamino)-N-(1-(2-m-ethyl-1-(neopentylamino)propan-2-yl)-1H-imidazol-4-yl)pentanamide; the CAS Registry Number is 865773−15-5; (commercially available from www.adooq.com)), RO-4929097 (RO4929097 refers to 2,2-dimethyl-N—((S)-6-oxo-6,7-dihydro-5H-dibenzo[b,diazepin-7-yl)-N-(2; 2,3,3,3-pentafluoro-propyl)-malonamide. The CAS Registry Number is 847925-91−1) (commercially available from www.adooq.com), MK-0752 (CAS No. 471905-41-6 (www.medchemexpress.com)); itanapraced (CAS No. 749269-83-8 (www.medchemexpress.com)); LY-3056480 (Samarajeewa, Anshula & Jacques, Bonnie & Dabdoub, Alain. (2019). Therapeutic Potential of Wnt and Notch Signaling and Epigenetic Regulation in Mammalian Sensory Hair Cell Regeneration. Molecular Therapy. 27. 10.1016/j.ymthe.2019.03.017); fosciclopirox (available as a disodium heptahydrate) (Patel, M. R., et al., Safety, dose tolerance, phatmacokinetics, and pharmacodynamics of fosciclopirox (CPX-POM) in patients with advanced solid tumors. Journal of Clinical Oncology (2020) 38:6 suppl 518); tarenflurbil (CAS No. 51543-40-9; (2R)-2-(3-fluoro-4-phenylphenyl)propanoic acid); EVP-0962 (Rogers, K., et al., (2012). Modulation of 7-secretase by EVP-0015962 reduces amyloid deposition and behavioral deficits in Tg2576 mice. Molecular Neurodegeneration. 7.61. 10.1186/1750-1326-7-61.; NICS-15; E-2212; GSI-1; NGP-555; PF-0664867); begacestat (aka GSI-953) (5-Chloro-N [(1S)-3,3,3-trifluoro-1-(hydroxymethyl)-2-(trifluoromethyl)propyl]-2-thiophenesulfonamide) (www.tocris.com); GSI-136 (5-chloro-N-[(2S)-3-ethyl-1-hydroxypentan-2-yl]thiophene-2-sulfonamide) (https://pubchem.ncbi.nlm.nih.gov/compound/gsi-136); and BMS-708163 (Gillman, K W et al, Discovery and Evaluation of BMS-708163, a Potent, Selective and Orally Bioavailable Gamma-Secretase Inhibitor. ACS Med. Chem. Lett. (2010) 1(3)120-124). See also, Sekioka, R. et al., Discovery of N-ethylpyridine-2-carboxamide derivatives as a novel scaffold for orally active gamma secretase modulators. Bioorg. & Med. Chem., (2020) 28(1): 115132. “Carboxamide-based GSI” means a GSI having a carboxamide group, and includes molecules formed by carboxamide substitution as well as derivatives of known carboxamide-based GSIs such as DAPT. Examples include DAPT (N-[(3,5-Difluorophenyl)acetyl]-L-alanyl-2-phenyl]glycine-1,1-dimethylethyl ester), semagacestat, avagacestat ((2R).-2-[[(4-Chlorophenyl)sulfonyl][[2-fluoro-4-f 1,2,4-ioxadiazol-3-yl)pbcnyl]methyl]amino]-5,5,5-trifluoropentanamide) (commercially available from www.tocris.com). DBZ (:N-[(1S)-2-[[(75)-6,7-Dihydro-5-methyl-6-oxo-5H-di benz[b,d]azcpin-7-yl amino]-I-methyl-2-oxoethyl]-3.5-difluorobenzeneacet:unide) (commercially available from www.tocris.com), L-685,458 ((5S)-(ten-Butoxycattbonylanuno)-6-phenyl-(4Rl-hvdroxy-(2R)-benzvlhe; xanovl)-L-leucy-L-phenylalaninamide) (commercially available from www.tocris.com), BMS-906024 (bis(fluoroalkyl)-1,4-benzodiazepinone; CAS Registry Number 1401066-79-2), Crenigascestat (aka 1.Y3039478) (Massard et al., “First-in-human study of LY3039478, a Notch signaling inhibitor in advanced or metastatic cancer,” J Clin Oncol (2015) 33(15_suppl):2533). MRK 560 (1V-[cis-4-[(4-Chlorophenyl)sulfonyljj-4-(2.5-difluorophenyl cyclohexylj-1,l,l-trifluoromethanesulfonamide) (commercially available from www.tocris.com), nirogacestat (aka PF-03084014)((S)-2-((S)-5,7-difluoro-1,2,3,4-tetrahydronaphthalen-3-ylamino)-N-(1-(2-m-ethyl-1-(neopentylamino)propan-2-yl)-1H-imidazol-4-yl)pentanamide; the CAS Registry Number is 865773−15-5; (commercially available from www.adooq.com)), RO-4929097 (RO4929097 refers to 2,2-dimethyl-N—((S)-6-oxo-6,7-dihydro-5H-dibenzo[b,diazepin-7-yl)-N′-(2; 2,3,3,3-pentafluoro-propyl)-malonamide. The CAS Registry Number is 847925-91−1) (commercially available from www.adooq.com) and BMS-708163 (Gillman, K W et al, Discovery and Evaluation of BMS-708163, a Potent, Selective and Orally Bioavailable Gamma-Secretase Inhibitor. ACS Med Chem. Lett. (2010) 1(3)120-124). See also, Sekioka, R. et al., Discovery of N-ethylpyridine-2-carboxamide derivatives as a novel scaffold for orally active gamma secretase modulators. Bioorg. & Med. Chem., (2020) 28(1): 115132. GSIs include any salt form, polymorph, hydrate, analog, or pro-drug that retains gamma secretase inhibiting or modulating activity.

“Treat,” “treatment.” “prevent,” “prevention.” “inhibit” and corresponding terms include therapeutic treatments, prophylactic treatments, and ones that reduce the risk that a subject will develop a disorder or risk factor. Treatment does not require complete curing of disorder or condition, and includes the reduction in severity, reduction in symptoms, reduction of other risk factors associated with the condition and/or disease modifying effects such as slowing the progression of the disease.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described. All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

### DETAILED DESCRIPTION

### Gamma Secretase Inhibitors

A variety of GSIs have been developed as potential clinical candidates for Alzheimer's Disease and cancer indications. (See Kreft et al, at 6171). DAPT was one of the earliest GSIs identified.

Modifications of DAPT led to clinical candidates.

Semagacestat is (2S)-2-hydroxy-3-methyl-N-[(1 S)-1-methyl-2-oxo-2-[[(1 S)-2,3,4,5-tetrahydro-3-methyl-2-oxo-1H-3-benzazepin-1-yl]amino]ethyl]-butamide, a small molecule gamma secretase inhibitor that was initially developed for the treatment of Alzheimer's Disease. (See U.S. Pat. No. 7,468,365). Semagacestat is known to exist in a number of polymorphic forms, including a dihydrate and at least two anhydrite forms. (Id., See also U.S. Pat. No. 8,299,059). See also, Yi et al, DMD (2010) 38:554-565; http://doi:10.1124/dmd.109.030841. Nirogacestat (aka PF-03084014) is ((S)-2-((S)-5,7-difluoro-1,2,3,4-tetrahydronaphthalen-3-ylamino)-N-(1-(2-m-ethyl-1-(neopentylamino)propan-2-yl)-1H-imidazol-4-yl)pentanamide, a small molecule gamma secretase inhibitor that was developed for cancer indications. It is available as a hydrobromide salt (www.medchemexpress.com), and exists is solid state forms. (See U.S. Pat. No. 10,590,087). See Wei P, et al. Evaluation of selective gamma-secretase inhibitor PF-03084014 for its antitumor efficacy and gastrointestinal safety to guide optimal clinical trial design. Mol Cancer Ther. 2010 June; 9(6):1618-28; and Kumar, S., et al., Clinical Activity of the gamma-secretase inhibitor PF-03084014 in adults with desmoid tumors (aggressive fibromatosis). J. Clin Oncol. (2017) May 10; 35(14):1561-1569. Seventeen patients were dosed at 50 mg orally twice a day in 3-week cycles for six cycles (18 weeks).

MK-0752 is a small molecule gamma-secretase inhibitor being studied for cancer indications. Phase 1 clinical data is described in Krop I, et al. Phase I pharmacologic and pharmacodynamic study of the gamma secretase (Notch) inhibitor MK-0752 in adult patients with advanced solid tumors. J Clin Oncol. 2012; 30(19):2307-2313. In this study, of 103 patients who received MK-0752, 21 patients received a continuous once-daily dosing at 450 and 600 mg; 17 were dosed on an intermittent schedule of 3 of 7 days at 450 and 600 mg; and 65 were dosed once per week at 600, 900, 1,200, 1,500, 1,800, 2,400, 3,200, and 4.200 mg. The most common drug-related toxicities were diarrhea, nausea, vomiting, and fatigue. Toxicity was found to be schedule dependent, with weekly dosing deemed generally well-tolerated. See also, Matthews et al, Journal of Chromatography B, 863 (2008) 36-45; https://doi:10.1016/j.jchromb.2007.12.025.

RO-4929097 is a small molecule gamma secretase inhibitor being studied for cancer indications. See, e.g., Tolcher A W, Messersmith W A, Mikulski S M et al. Phase I study of R04929097, a gamma secretase inhibitor of Notch signaling, in patients with refractory metastatic or locally advanced solid tumors. J Clin Oncol 2012; 30: 2348-2353; Wu et al, Journal of Chromatography B, 879 (2011) 1537 1543. In this study, patients received escalating doses of R04929097 orally on two schedules: (A) 3 consecutive days per week for 2 weeks every 3 weeks; (B) 7 consecutive days every 3 weeks; and (C) continuous daily dosing. Toxicities included fatigue, thrombocytopenia, fever, rash, chills, and anorexia. The study concluded that R04929097 was well tolerated at 270 mg on schedule A and at 135 mg on schedule B; and the safety of schedule C was not fully evaluated.

Crenigascestat (aka LY3039478) is a small molecule gamma secretase inhibitor being studied for cancer indications. See Yuen E., et al., Evaluation of the effects of an oral notch inhibitor, crenigacestat (Ly3039478), on QT interval, and bioavailability studies conducted in healthy subjects. Cancer Chemother PHarmacol. 2019 March; 83(3):483-492. In this study crcnigacestat was administered to healthy subjects as single 25.50, or 75 mg oral doses or as an intravenous dose of 350 μg ¹³C¹⁵N²H-crenigacestat.

In a Phase III trial in Alzheimer's Disease, semagacestat, dosed at 100 mg and 140 mg daily, did not improve cognitive status and patients on the highest dose showed a significant worsening of cognitive ability. Semagacestat was also associated with more adverse events, including skin cancers and infections. Doody, R. S., et al., N Engl J Med 369; 4: 341-350 (Jul. 25, 2013). An earlier Phase I study reported subjects dosed with 5 mg, 20 mg or 40 mg daily for 14 days showed adverse events similar to placebo, while 2 of 7 subjects receiving a 50 mg daily dose reported adverse events that may have been drug related. Siemens E. Skinner M, Dean R A, et al. Safety, tolerability, and changes in amyloid beta concentrations after administration of a gamma-secretase inhibitor in volunteers. Clin Neuropharmacol. 2005; 28(3):126-132.

Because of evidence that Notch-signaling is dysregulated in numerous malignancies, GSIs have been developed as potential cancer therapeutics, as monotherapies or in combination with other agents. See, e.g., Takebe N. Nguyen D, Yang S X. Targeting notch signaling pathway in cancer: clinical development advances and challenges. Pharmacol Ther. 2014 February; 141(2):140-9. doi: 10.1016/j.phannthera.2013.09.005. Epub 2013 Sep. 27; Shao H, Huang Q, Liu Z J. Targeting Notch signaling for cancer therapeutic intervention. Adv Pharmacol. 2012; 65:191-234. doi: 10. 101&B978-0-12-397927-8.00007-5.

It has now been found that low dose GSIs are effective in the treatment of respiratory diseases characterized by mucus hypersecretion, and are effective at doses allowing therapeutic activity while avoiding or minimizing the adverse effects previously associated with this class of molecules. Adverse effects with the use of GSIs generally involve adverse gastrointestinal events. Adverse gastrointestinal events include, without limitation, decreased appetite, nausea, vomiting, weight loss, diarrhea, gastrointestinal bleeding, etc. As used herein, “low dose” of a CSI refers to a dose that is an effective amount to treat the respiratory disease characterized by mucus hypersecretion and is a lower dose as compared to a dose of the GSI suitable for administering to a patient suffering from a neurodegenerative disorder, an oncology disorder, or a respiratory disease not characterized by mucus hyper-secretion. For example, a low dose would be a dose yielding a peak plasma level in the submicromolar range. It will be appreciated that a low dose of a GSI may be administered as a single daily dose, multiple doses per day (e.g., 2 or 3 doses per day), intermittent, or weekly, with the dosing regimen dependent on the dosage form (e.g., immediate release or controlled release), and the needs of the patient. Administration may be for an extended period of time, intermittent, or may be for a limited amount of time, with administration repeated if and to the extent determined by a patient's medical provider. For example, a GSI may be provided daily for 1, 3, 5, 7, 10, 14, 18, 21, 24, 28 or 30 days, and then stopped. In some embodiments, the GSI is administered intermittently, such as every 3 days, or weekly. It will be appreciated that references to daily dosing amounts herein can be accomplished by dosing regimens other than daily, e.g., a weekly dose of 35 mg would correspond to a daily dose of 5 mg/day. Likewise, slow-release formulations, such as depots or patch formulations are known in the art and can be utilized to provide doses equivalent to the daily doses described herein. The GSI dosing regimen may be repeated if necessary.

For example, each of the GSIs semagacestat, nirogacestat (PF-03084014), RO-4929097 and MK-0752, when administered in the nanomolar range to human nasal epithelial cells is effective in blocking Notch signaling, driving differentiation towards MCCs, and rescuing conditions associated with excessive goblet cell mucus secretion. Hence, relatively low systemic levels of GSLS may provide effective treatment for respiratory conditions associated with mucus hypersecretion, while avoiding or minimizing adverse events observed with higher doses.

In some embodiments, the low dose of the GSI reduces mucus in the patient's lungs and does not result in adverse gastrointestinal events in the patient. In some embodiments, the low dose of the GSI reduces mucus accumulation in the patient's lungs and does not result in adverse gastrointestinal events in the patient. In some embodiments, the adverse gastrointestinal event that is not experienced by the subject is selected from the group consisting of decreased appetite, nausea, vomiting, weight loss, diarrhea, and gastrointestinal bleeding.

It has further been found that GSIs administered in combination with a CFTR modulator is effective in correcting epithelial cell dysfunction in cystic fibrosis cell-based model systems (primary cells from patients), in contrast to certain prevailing concepts, and indeed the combination may be synergistic in improving CFTR ion channel function and epithelial cell correction. For example, various GSIs have now been shown to not interfere with CFTR ion channels and not inhibit effects of CFTR modulators on CFTR ion channels in cystic fibrosis airway epithelial cells. It has been found that GSI treatment, surprisingly, improves airway surface liquid (ASL) reabsorption of CF cells to the same degree as CFTR modulator drugs. Further evidence suggests that GSI treatment may be synergistic with CFTR modulator treatment, thereby allowing the potential to decrease doses of either or both drugs, and further reducing potential toxicities of each.

GSIs may be administered by pharmaceutical dosage forms known in the art, including but not limited to oral solid dosages, oral liquids, injection, transdermal patch, and inhalation. Dosage forms may be formulated with excipients and other compounds to facilitate administration to a subject and to maintain shelf stability. See “Remington's Pharmaceutical Sciences” (Mack Publishing Co., Easton, PA). Oral pharmaceutical formulations include tablets, minitablets, pellets, granules, capsules, gels, liquids, syrups and suspensions. Preferably, a GSI is administered orally, typically via oral solid dosage, although oral liquids may be desirable for certain populations that have difficulty with tablets and capsules, such as pediatric and elderly patients. Oral dosage forms may be immediate release or controlled release.

Tablet forms of semagacestat are known in the art. (See U.S. Pat. No. 8,299,059). Upon oral administration, semagacestat is reported to have a half-life of approximately 2.5 hours. Hence, in one embodiment of the invention, semagacestat may be provided as an immediate release formulation. Immediate release semagacestat may be provided as a single daily dose, or divided into multiple daily dosages which may be administered 2, 3, 4 or more times per day. In another embodiment of the invention, semagacestat is provided as an extended release formulation. An extended release formulation may provide patient convenience by reducing daily administrations, and may improve patient compliance. Further, controlled release formulations of the present invention may be useful in reducing serum peaks and troughs, thereby potentially reducing adverse events.

Oral controlled release formulations are known in the art and include sustained release, extended release, delayed release and pulsatile release formulations. See “Remington's Pharmaceutical Sciences” (Mack Publishing Co., Easton, PA). The active agent may be formulated in a matrix formulation with one or more polymers that slow release of the drug from the dosage form, including hydrophilic or gelling agents, hydrophobic matrices, lipid or wax matrices and biodegradable matrices. The active agent may be formulated in the form of a bead, for example with an inert sugar core, and coated with known excipients to delay or slow release of the active agent by diffusion. Enteric coatings are known in the art for use in delaying release of an active agent until the dosage form passes from the low pH environment of the stomach to the higher pH environment of the small intestine, and my include methyl acrylate-methacrylic acid copolymers, cellulose acetate phthalate (CAP), cellulose acetate suecinate, hydroxypropyl tnethylcellulose phthalate, hydroxypropyl methylcellulose acetate succinate, polyvinyl acetate phthalate (PVAP), shellac, sodium alginate, and cellulose acetate trimellitate.

In some embodiments, the GSI is selected from the group consisting of semagacestat, avagacestat, GS-1, DBZ.1.685,458, BMS-906024, crenigascestat, MRK 560, nirogacestat, RO-4929097, MK-0752, itanapraced, LY-3056480, fosciclopirox, tarenflurbil, and begacestat.

In some embodiments, the GSI is selected from semagacestat., nirogacestat, MK-0752, RO-492907, or crenigacestat. In one embodiment, the GSI is semagacestat.

In some embodiments, a method is provided for treating a respiratory disease characterized by mucus hyper-secretion comprising systemically administering to a patient in need thereof about 0.1 mg to about 50 mg semagacestat daily wherein the oral administration of semagacestat is effective in reducing mucus in such patient's lungs or inhibiting mucus accumulation in such patient's lungs. Preferably, semagacestat is systemically administered at dosages of from about 0.5 mg to about 40 mg daily, or from about 0.5 mg to about 30 mg daily, and most preferably of from about 0.5 mg to about 20 mg daily, or from 0.5 mg to about 10 mg daily. For example, semagacestat may be administered in about 0.1 mg. 0.25 mg, 0.5 mg, 1 mg, 2.5 mg, 5 mg, 10 mg, 15 mg, 20 mg, 25 mg, 30 mg, 35 mg. 40 mg. 45 mg or 50 mg daily.

In another embodiment, methods are provided for treating a respiratory disease characterized by mucus hypersecretion comprising systemically administering to a patient in need thereof about 5 μg to about 1 mg/kg daily, preferably from about 50 to about 100 μg/kg semagacestat daily.

In an embodiment of the invention, a method is provided for treating a respiratory disease characterized by mucus hyper-secretion comprising systemically administering to a human patient in need of such treatment a therapeutically effective amount of semagacestat, wherein said patient's semagacestat plasma concentration at steady state following multiple dose administration comprises an AUC (area under the curve) less than 1220 ng·hr/mL wherein the systemic administration of semagacestat is effective in reducing mucus in such patient's lungs or inhibiting mucus accumulation in such patient's lungs. In some embodiments, upon multiple dose administration, said patient's steady state semagacestat plasma concentration comprises an AUC less than 1220 ng·hr/mL, less than 600 ng·hr/mL, or less than 250 ng·hr/mL. In some embodiments, the patient's semagacestat plasma concentration at steady state following multiple dose administration comprises an AUC less than 1220 ng·hr/mL and the patient has not experienced an adverse gastrointestinal event.

In some embodiments, a method is provided for treating a respiratory disease characterized by mucus hyper-secretion comprising systemically administering to a patient in need thereof about 0.1 mg to about 50 mg nirogacestat daily wherein the oral administration of nirogacestat is effective in reducing mucus in such patient's lungs or inhibiting mucus accumulation in such patient's lungs. In some embodiments the nirogacestat is systemically administered at dosages of from about 0.5 mg to about 40 mg daily, or from about 0.5 to about 30 mg, or of from about 0.5 mg to about 20 mg daily.

In another embodiment, methods are provided for treating a respiratory disease characterized by mucus hypersecretion comprising systemically administering to a patient in need thereof about 8 μg to about 0.9 mg/kg daily, preferably from about 10 to about 300 μg/kg nirogacestat daily. In some embodiments, a method is provided for treating a respiratory disease characterized by mucus hyper-secretion comprising systemically administering to a patient in need thereof about 0.1 mg to about 20 mg RO-4929097 daily wherein the oral administration of RO-4929097 is effective in reducing mucus in such patient's lungs or inhibiting mucus accumulation in such patient's lungs. In some embodiments the RO-4929097 is systemically administered at dosages of from about 0.1 to about 10 mg daily, or from about 0.5 mg to about 10 mg daily, or of from about 0.1 mg to about 5 mg daily.

In another embodiment, methods are provided for treating a respiratory disease characterized by mucus hypersecretion comprising systemically administering to a patient in need thereof about 5 μg to about 0.4 mg/kg daily, preferably from about 50 to about 100 μg/kg RO-4929097 daily.

In some embodiments, a method is provided for treating a respiratory disease characterized by mucus hyper-secretion comprising systemically administering to a patient in need thereof about 0.1 mg to about 40 mg MK-0752 daily wherein the oral administration of MK-0752 is effective in reducing mucus in such patient's lungs or inhibiting mucus accumulation in such patient's lungs. In some embodiments, the MK-0752 is administered at a dose in the range of from about 0.1 to about 30 mg daily, or from about 0.1 mg to about 20 mg daily, and most preferably of from about 0.1 mg to about 10 mg daily.

In another embodiment, methods are provided for treating a respiratory disease characterized by mucus hypersecretion comprising systemically administering to a patient in need thereof about 2.5 μg to about 0.6 mg/kg daily, preferably from about 2.5 to about 500 μg/kg MK-0752 daily.

In some embodiments, the respiratory disease characterized by mucus hypersecretion is selected from the group consisting of cystic fibrosis, chronic obstructive pulmonary disease, primary ciliary dyskinesia, chronic bronchitis, asthma, idiopathic and secondary bronchiectasis, bronchiolitis obliterans, Idiopathic pulmonary fibrosis and other fibrotic lung disorders and respiratory infection, including exacerbations in chronic respiratory disorders and mucus accumulation in response to acute infection. In some embodiments, the respiratory disease is cystic fibrosis. In other embodiments, the respiratory disease is chronic obstructive pulmonary disease.

In some embodiments, the GSI is administered locally. In some embodiments, the local administration is administered by inhalation. In some embodiments, the GSI is administered by inhalation. In some embodiments, the GSI is administered systemically. In some embodiments, the systemic administration is administered by oral administration. In preferred embodiments, the GSI is administered by oral administration. In one embodiment, the GSI is provided in an immediate release solid oral dosage form. In another embodiment, the GSI is provided in a controlled release solid oral dosage from. In a further embodiment, the GSI is provided in a liquid dosage form. In a further embodiment, the GSI is provided in an inhalation dosage form.

### Cystic Fibrosis Combination Therapy

CFTR modulator drugs have provided a significant advance in the treatment of cystic fibrosis. They do not, however, address damage that occurs to the lung epithelium due to cystic fibrosis. Further, CFTR modulators are limited to use in patients that have the specific CFTR mutations addressed by the particular CFTR modulator drug.

The cell type or types that most express functional CFTR is not defined. It has been suggested that a rare cell type named ionocyte might be the major source of CFTR expression and therefore activity. Plasschaert, L. W., et. al., A single-cell atlas of the airway epithelium reveals the CFTR-rich pulmonary ionocyte. Nature (2018) 560: 377-381; https:/doi.org/10.1038/s41586-018-0394-6. Furthermore, it was suggested that the ionocyte population is expected to be diminished upon Notch inhibition and CFTR activity likewise decrease. (Id. at 380). Other data published more recently suggests that a diversity of cell types express varying levels of CFTR Carraro, G., et al., Nat. Med. (2021) May; 27(5):806-814. Doi: 10. 1038/s41591-021-01332-7.Epub 2021 May 5. In contrast to published assertions of GSI interference with CFTR activity, it has now been found that GSI treatment does not diminish CFTR activity. Rather, it has been found that GSI treatment, surprisingly, improves ciliary beat frequency (CBF) and mucus transport of CF cells to the same degree as CFTR modulator drugs. Further evidence suggests that GSI treatment may be synergistic with CFTR modulator treatment, thereby allowing the potential to decrease doses of either or both drugs, and further reducing potential toxicities of each.

Accordingly, methods of the invention address the dysfunction present in cystic fibrosis airway and other epithelial cells that lead to mucus hypersecretion (and often infection), by promoting differentiation of MCCs and reduction of mucus secreting cells, and enabling improved mucociliary clearance. Administration of a GSI may improve epithelial function in cystic fibrosis without regard to the CFTR mutations causing the underlying disease. Hence, in the treatment of cystic fibrosis, a GSI may be administered alone or in combination with any CFTR modulator or combination of CFTR modulators.

In some embodiments of the invention, methods are provided for treating cystic fibrosis comprising administering to a patient in need thereof a therapeutically effective amount of a GSI and a CFTR modulator. The GSI may be administered prior to, after or concurrently with the CFTR modulator. In some embodiments, the CSI is administered orally to a patient taking a CFTR modulator. In some embodiments, the GSI is administered by inhalation to a patient taking a CFTR modulator. The CSI may be provided in a single course of treatment or may be provided intermittently in combination with a CFTR modulator dosing regimen.

For example, a CFTR modulator may be administered daily and a GSI may be administered daily for 1, 3, 5, 7, 10, 14, 18, 21, 24, 28 or 30 days, and then stopped. In some embodiments, the GSI is administered intermittently, such as every 3 days, or weekly. It will be appreciated that references to daily dosing amounts herein can be accomplished by dosing regimens other than daily, e.g., a weekly dose of 35 mg would correspond to a daily dose of 5 mg/day. Likewise, slow-release formulations, such as depots or patch formulations are known in the art and can be utilized to provide doses equivalent to the daily doses described herein. The GSI dosing regimen may be repeated if necessary. In some embodiments, the GSI is selected from semagacestat, nirogacestat, MK-0752, RO-492907, or crenigacestat. In one embodiment, the GSI is semagacestat.

CFTR modulators useful in the present invention include CFTR potentiators, correctors, premature stop codon suppressors, amplifiers and combinations thereof. Currently marketed CFTR modulators include ivacaftor, lumacaftor, tezacaftor and elexacaftor and combinations. Ivacaftor is marketed in tablet and granule form as KALYDECO. (See U.S. Pat. Nos. 7,495,103 and 8,754,224). Ivacaftor and tezacaftor are marketed as SYMDEKO. (See U.S. Pat. Nos. 7,745,789; 7,776,905; 8,623,905 and 10,239,867). A combination of lumacaftor and ivacaftor is marketed as ORKAMBI. (See U.S. Pat. Nos. 8,507,534 and 10,597,384). A combination of elexacaftor, ivacaftor and tezacaftor (“ETI”) is marketed as TRIKAFTA. Additional CFTR modulators that may be used in the present invention are in development. (See, e.g., U.S. Pat. Nos. 10,647,717; 10,604,515; 10,568,867; 10,428,017; 10,399,940; 10,259,810; 10,118,916; 9,895,347; 10,550,106; 10,548,878; 10,392,378; 10,494,374; 10,377,762; 10,450,273; 9,890,149 and 10,258,624).

In an embodiment of the invention, methods are provided for treating cystic fibrosis by administration of a therapeutically effective amount of a GSI and a CFTR modulator. In another embodiment, a method of treating cystic fibrosis is provided in which a GSI is systemically administered to a patient being administered or in need of administration of a CFTR modulator. The GSI may be provided concurrently, prior to or after administration of the CFTR modulator. In some embodiments, the GSI is provided intermittently in combination with a CFTR modulator dosing regimen.

In one embodiment, a method of treating cystic fibrosis in a patient being administered or in need administration of a CFTR modulator is provided, comprising systemically administering to such patient about 0.1 mg to about 50 mg semagacestat daily wherein the oral administration of semagacestat is effective in reducing mucus in such patient's lungs or inhibiting mucus accumulation in such patient's lungs. In some embodiments, semagacestat is systemically administered at dosages of from about 0.5 mg to about 40 mg daily. In some embodiments, semagacestat is administered at a dosage of from about 0.5 mg to about 20 mg daily.

In one embodiment, a method of treating cystic fibrosis in a patient taking a CFTR modulator is provided comprising systemically administering semagacestat to a patient in need thereof about 5 μg to 1 mg/kg daily, preferably from about 50 to 100 μg/kg daily.

In some embodiments, semagacestat is provided orally to a patient taking a CFTR modulator wherein said patient's semagacestat plasma concentration at steady state following multiple dose administration comprises an AUC (area under the curve) less than 1220 ng·hr/mL, wherein the systemic administration of semagacestat is effective in reducing mucus in such patient's lungs or preventing mucus accumulation in such patient's lungs. In some embodiments, upon multiple dose administration, said patient's steady state semagacestat plasma concentration comprises an AUC less than 1220 ng·hr/mL, less than 600 ng·hr/mL, or less than 250 ng·hr/mL. In some embodiments, the patient's semagacestat plasma concentration at steady state following multiple dose administration comprises an AUC less than 1220 ng·hr/mL and the patient has not experienced an adverse gastrointestinal event. Steady state semagacestat levels may be determined following about 1 week or about 2 weeks or more of administering the therapeutically effective amount of semagacestat.

In some embodiments, a method of treating cystic fibrosis in a patient being administered or in need administration of a CFTR modulator is provided comprising systemically administering to a patient in need thereof about 0.1 mg to about 50 mg nirogacestat daily wherein the oral administration of nirogacestat is effective in reducing mucus in such patient's lungs or inhibiting mucus accumulation in such patient's lungs. In some embodiments the nirogacestat is systemically administered at dosages of from about 0.5 mg to about 40 mg daily, or from about 0.5 to about 30 mg, or of from about 0.5 mg to about 20 mg daily.

In another embodiment, a method of treating cystic fibrosis in a patient taking a CFTR modulator is provided comprising systemically administering to a patient in need thereof about 8 μg to about 0.9 mg/kg daily, preferably from about 10 to about 300 μg/kg nirogacestat daily.

In some embodiments, a method of treating cystic fibrosis in a patient taking a CFTR modulator is provided comprising systemically administering to a patient in need thereof about 0.1 mg to about 20 mg RO-4929097 daily wherein the oral administration of RO-4929097 is effective in reducing mucus in such patient's lungs or inhibiting mucus accumulation in such patient's lungs. In some embodiments the RO-4929097 is systemically administered at dosages of from about 0.1 to about 10 mg daily, or from about 0.5 mg to about 10 mg daily, or of from about 0.1 mg to about 5 mg daily.

In another embodiment, a method of treating cystic fibrosis in a patient taking a CFTR modulator is provided comprising systemically administering to a patient in need thereof about 5 μg to about 0.4 mg/kg daily, preferably from about 50 to about 100 μg/kg RO-4929097 daily.

In some embodiments, a method of treating cystic fibrosis in a patient taking a CFTR modulator is provided comprising systemically administering to a patient in need thereof about 0.1 mg to about 40 mg MK-0752 daily wherein the oral administration of MK-0752 is effective in reducing mucus in such patient's lungs or inhibiting mucus accumulation in such patient's lungs. In some embodiments, the MK-0752 is administered at a dose in the range of from about 0.1 to about 30 mg daily, or from about 0.1 mg to about 20 mg daily, and most preferably of from about 0.1 mg to about 10 mg daily.

In another embodiment, a method of treating cystic fibrosis in a patient taking a CFTR modulator is provided comprising systemically administering to a patient in need thereof about 2.5 μg to about 0.6 mg/kg daily, preferably from about 2.5 to about 500 μg/kg MK-0752 daily.

In some embodiments, the GSI is administered locally. In some embodiments, the local administration is administered by inhalation. In some embodiments, the GSI is administered by inhalation. In some embodiments, the GSI is administered systemically. In some embodiments, the systemic administration is administered by oral administration. Preferably, the GSI is provided by oral administration. The GSI may be provided as an immediate release oral dosage form, or as a controlled release oral dosage form.

Generally, the human subject that is treated by methods of the invention. e.g., as described above, is one that has been diagnosed as having a respiratory disease characterized by mucus hypersecretion. In some instances, the respiratory disease characterized by mucus hypersecretion for which the subject is diagnosed as having is selected from the group consisting of cystic fibrosis, chronic obstructive pulmonary disease, primary ciliary dyskinesia, chronic bronchitis, asthma, idiopathic and secondary bronchiectasis, bronchiolitis obliterans, Idiopathic pulmonary fibrosis and other fibrotic lung disorders and respiratory infection, including exacerbations in chronic respiratory disorders, and mucus accumulation in response to acute infection. In some embodiments, the subject is a subject diagnosed as having cystic fibrosis. In other embodiments, the subject is a subject diagnosed as having chronic obstructive pulmonary disease.

The following examples are offered by way of illustration and not by way of limitation.

### EXAMPLES

### Example 1: Treatment of HNECs with GSIs

Air-Liquid Interface (ALI) cultures were prepared as described in Vladar E K, Nayak J V, Milla C E, Axelrod J D. Airway epithelial homeostasis and planar cell polarity signaling depend on multiciliated cell differentiation. JCI Insight. 2016; 1(13); e88027. Human nasal epithelial cells (HNECs) were generated from human sinonasal epithelial brushings or from tissue obtained from patients undergoing endoscopic sinus surgery at Stanford Hospital and cultured as described in Vladar et al.

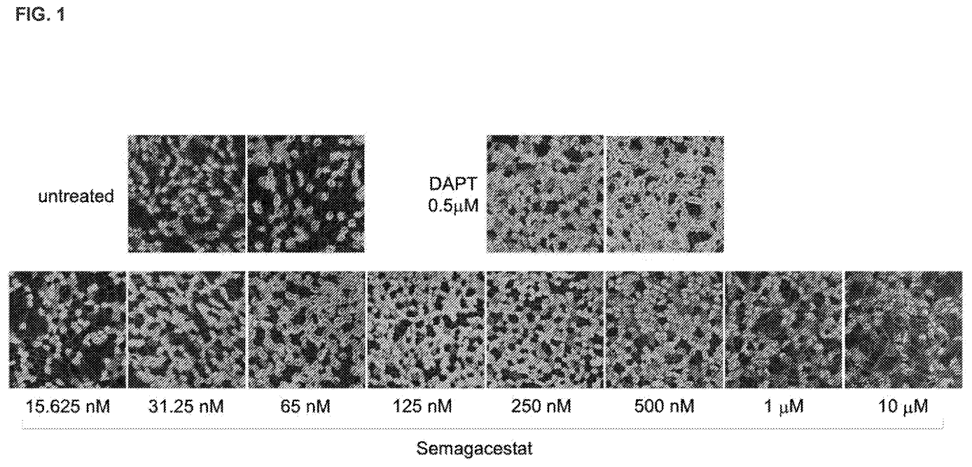

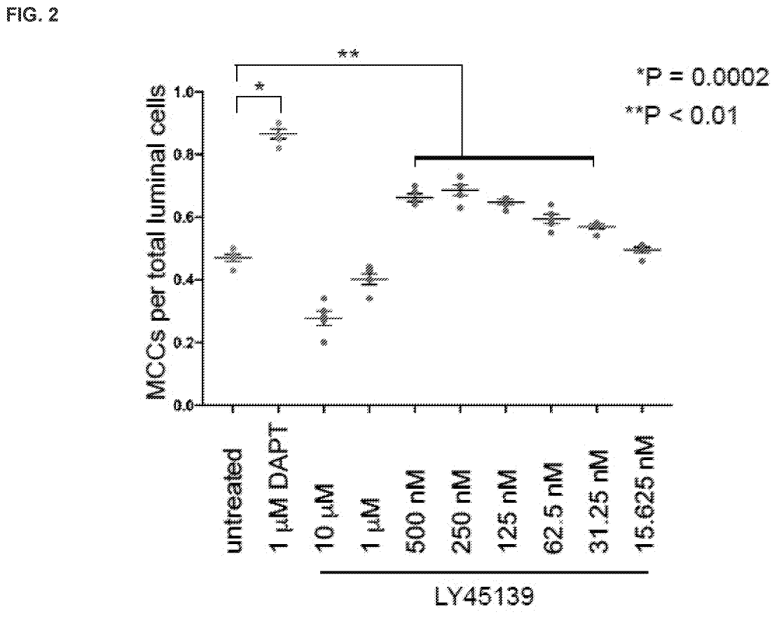

Cultures were treated with varying doses of semagacestat. DAFT (Abcam) was used (0.5 μg) as a positive cone ml for GSI activity. Cultures were labeled at ALI+21 d with anti-acetylated α-tubulin (green) and ECAD (red) antibodies to mark cilia and epithelial junctions. Results shown in FIG. 1 demonstrate dose response of HNECs treated with semagacestat, and show effective conversion to MCCs with nanomolar Using ECAD, we counted the total number of cells, and anti-acetylated α-tubulin was used to count the number of MCCs (does not include immature MCCs that have been fated but have not made cilia yet, as our focus was on “functional” MCCs). The ratio was defined as MCC number/total luminal cells. Counting was done on one representative image from 5 culture replicates from a single donor. Results shown in FIG. 2 show that both I μM DAPT and semagacestat in doses ranging from 500 nM to 31.25 nM produced significant increases in this ratio (all p<0.01 by ANOVA with pct hoc Dunnett's multiple comparisons test).

The effective semistatic doses correspond to more than two curlers of magnitude lower than those used in human Alzheimer's Disease trials.

### Example 2: In Vivo Mouse Models

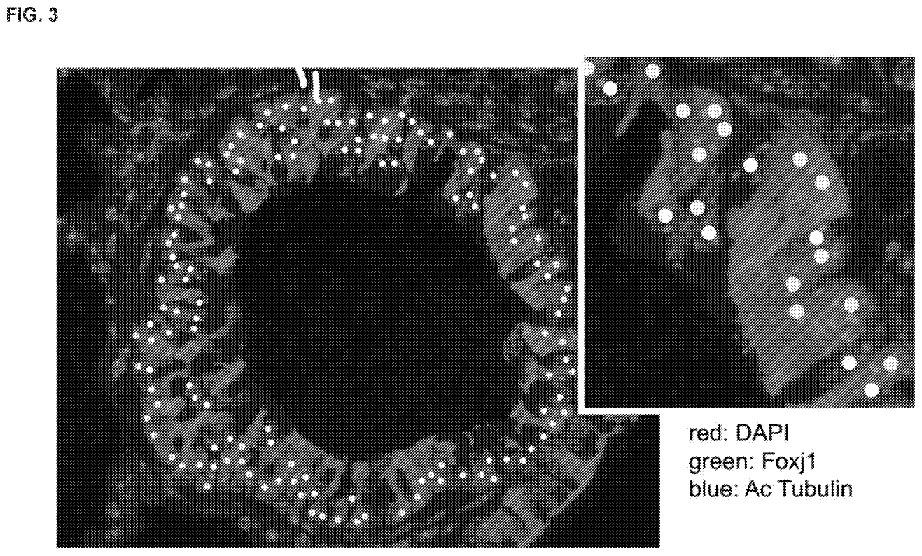

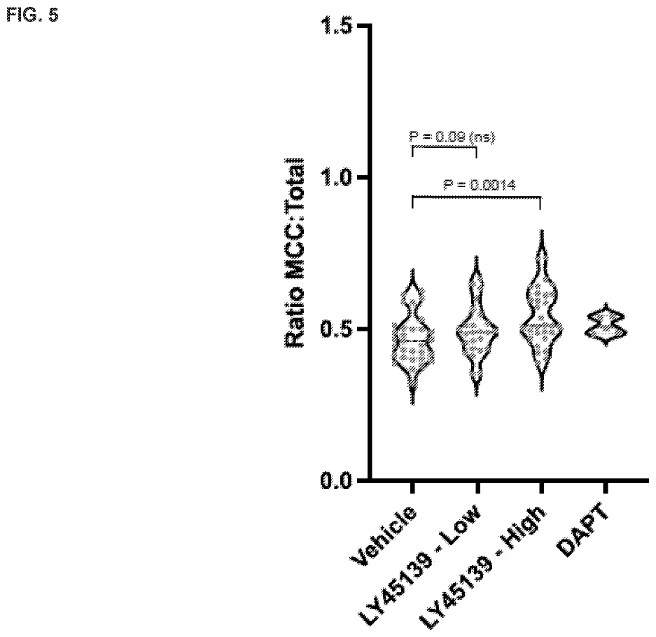

10-40 week age-matched male and female Foxj1-GFP mice were given semagacestat or vehicle by intraperitoneal (IP) administration. In the first experiment, semagacestat was administered at 0.1 mg/kg and 1 mg/kg twice daily for three days and compared to vehicle alone. Mice were sacrificed on day 7. FIG. 3 demonstrates the method of evaluating airway cellular composition. In approximately similar sized, PFA-fixed airways, nuclei (red; DAPI) were scored either as MCCs (green; GFP) or non-ciliated cells (absence of GFP). Acetylated tubulin (blue) marks cilia. Samples were blinded for treatment group prior to scoring. FIG. 5 shows an increase in the ratio of ciliated to non-ciliated cells following 3 days of systemic (IP) semagacestat treatment at both the low and high doses.

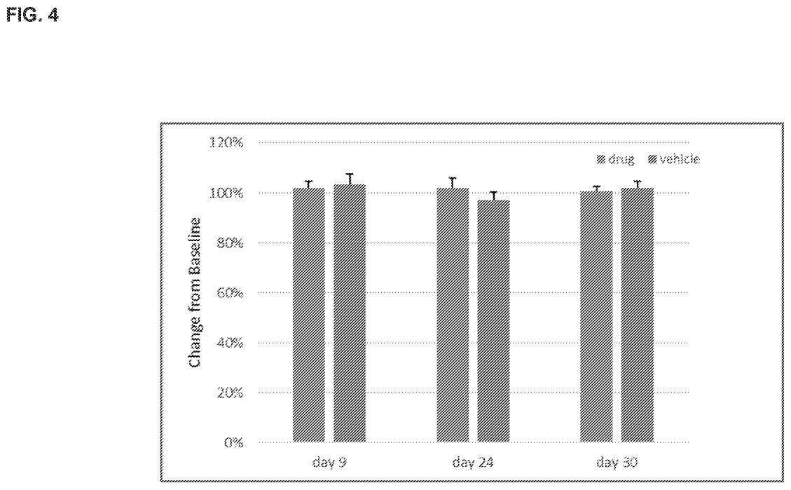

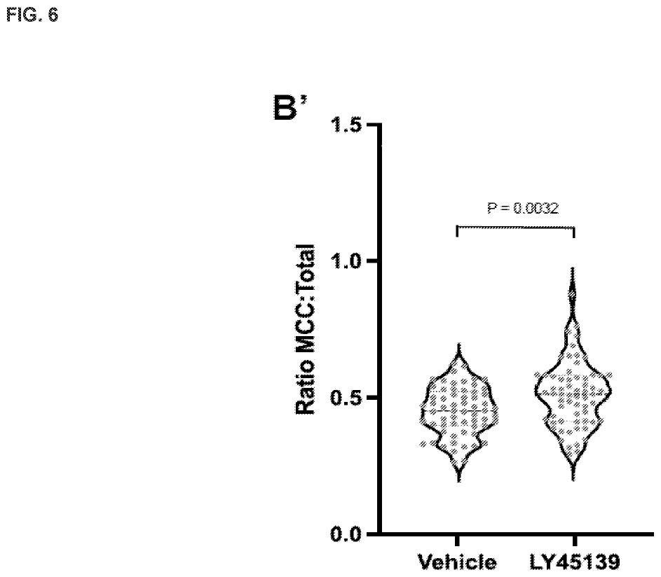

In the second experiment, vehicle and semagacestat were administered once daily for 5 consecutive days per week for three weeks, with a semagacestat dose of 1 mg/kg. An important observed toxicity in multiple GSI clinical trials, including large Alzheimer's Disease trials, was gastrointestinal toxicity. As a surrogate measure of GI toxicity, we monitored body weight throughout the experiment. Body weight was measured on Days 1, 9, 24 and 30, and mice were sacrificed on day 31. No mortality or ill effects were noted in any group. FIG. 4 shows body weight at days 9, 24 and 30 and demonstrates no significant difference in treatment groups as compared to vehicle controls. FIG. 6 shows the significant increase in the ratio of ciliated to non-ciliated cells after 3 weeks of systemic (IP) semagacestat.

In the three-day treatment, a dose response trend was observed, with the high dose reaching statistical significance (FIG. 5). The three-week treatment response at higher dose once a day was highly significant (FIG. 6).

### Example 3: Dependence of Multiciliated Cell Formation on Timing of Treatment in Differentiating and Mature Airway Epithelia

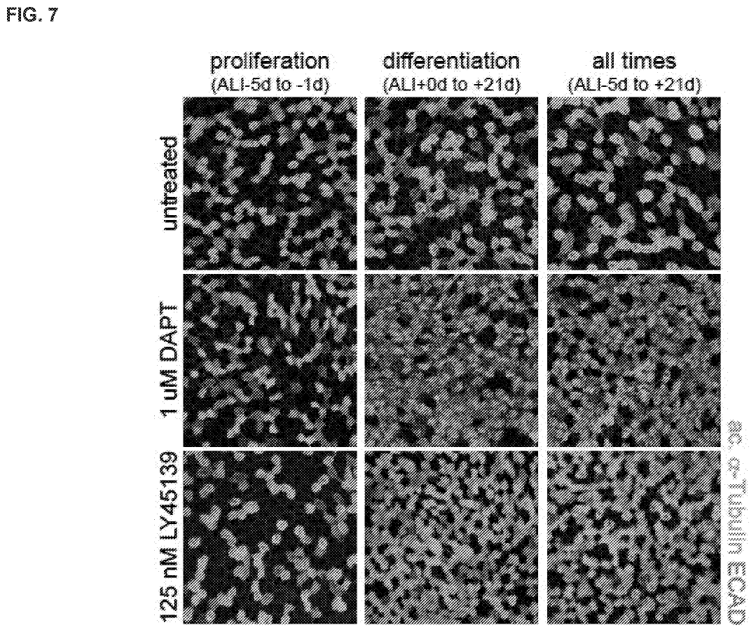

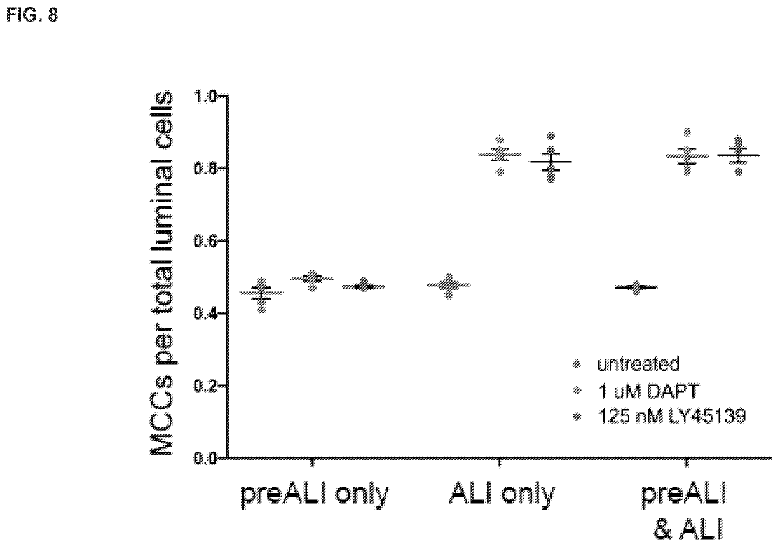

Primary human airway epithelial cells were treated with DAPT and LY45139: i) during proliferation only (ALI-5 to -Id), ii) during differentiation only (ALI+0 to +21d) or iii) continuously during the entire culture duration, followed by labeling at ALI+21d with anti-acetylated α-Tubulin (green) and ECAD (red) antibodies. GSI treatment during multiciliated cell differentiation (differentiation only and continuous treatments) increased MCC cell numbers. Results shown in FIG. 7 show that GSI treatment during proliferation only had no effect on differentiation, nor a detrimental effect on subsequent differentiation or overall epithelial structure. FIG. 8 shows quantitation (MCCs per total luminal cells) of data shown in FIG. 7.

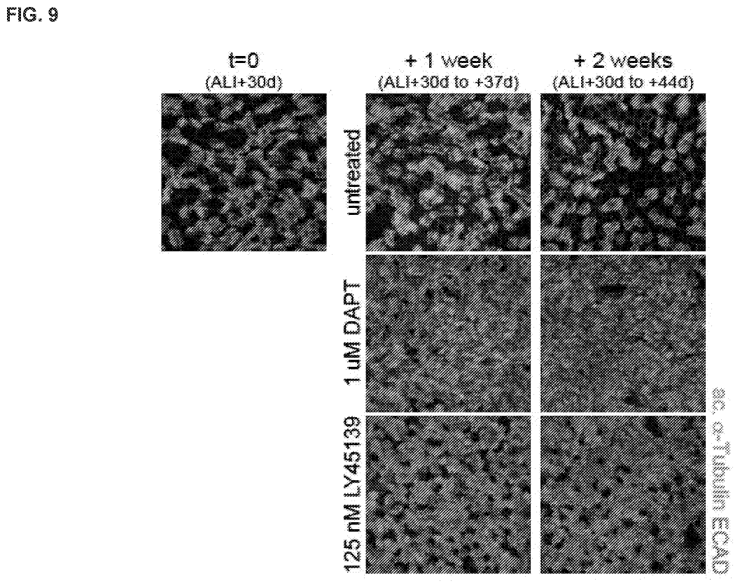

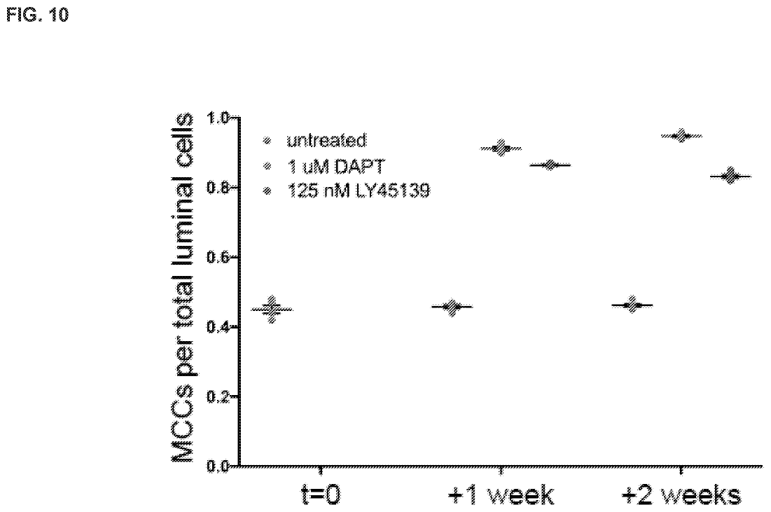

Mature (ALI+30d) primary human airway epithelial cells were treated with DAFT and semagacestat for one (ALI+30 to +37d) or two weeks (ALI+30 to +44d), then labeled with anti-acetylated α-Tubulin (green) and ECAD (red) antibodies. Results shown in FIG. 9 show that GSI treatment induces the formation of additional multiciliated cells in mature cultures, while untreated cultures do not differentiate any more multiciliated cells. FIG. 10 shows the quantitation of data shown in FIG. 9.

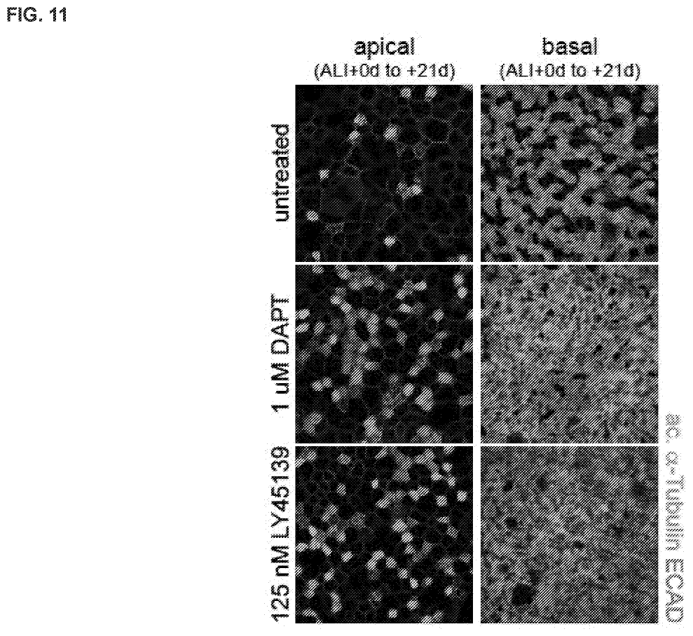

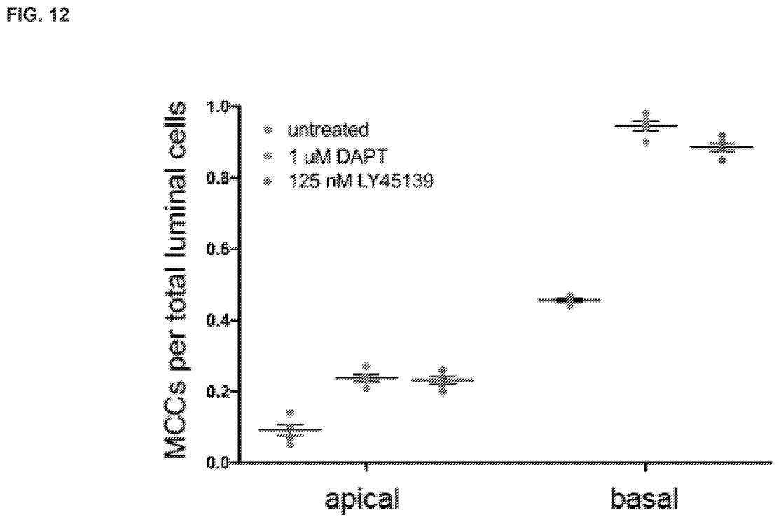

Primary human airway epithelial cells were treated with DAFT and LY45139 during differentiation only (ALI+0 to +21d) from either the apical or basal surface, then labeled at ALI+21d with anti-acetylated α-Tubulin (green) and ECAD (red) antibodies. Results shown in FIG. 11 show that GSI treatment induces ciliated cell formation via both apical and basal application. Apical treatment eliminates the air-liquid interface, which results in the poor epithelial structure and multiciliated cell differentiation in the untreated cultures, which is partially rescued by GSI treatment. FIG. 12 shows quantitation of data shown in FIG. 11. This result suggests that both systemic exposure and inhalation exposure are likely to be effective in vivo.

The results show that treatment during or after differentiation increases the ratio of MCCs to total cells. Treatment during proliferation (prior to differentiation) has no apparent effect, either beneficial or adverse.

### Example 4: IL-13 Induced Chronic Inflammation Model

Administration of IL-13 to ALI cultures induces goblet cell hyperplasia and is a useful model of chronic inflammation. ALI HNEC cultures were prepared as described in Vladar et al. ALI cultures were treated with and without administration of I1-13 on days 7−14. DAFT (1 um), semagacestat (125 nm) or vehicle control were administered on days 14-21. PFA-fixed cultures were stained for Muc5AC (red; mucin producing secretory cells), Acetylated tubulin (green; MCCs) and E-Cadherin (blue to reveal cell boundaries).

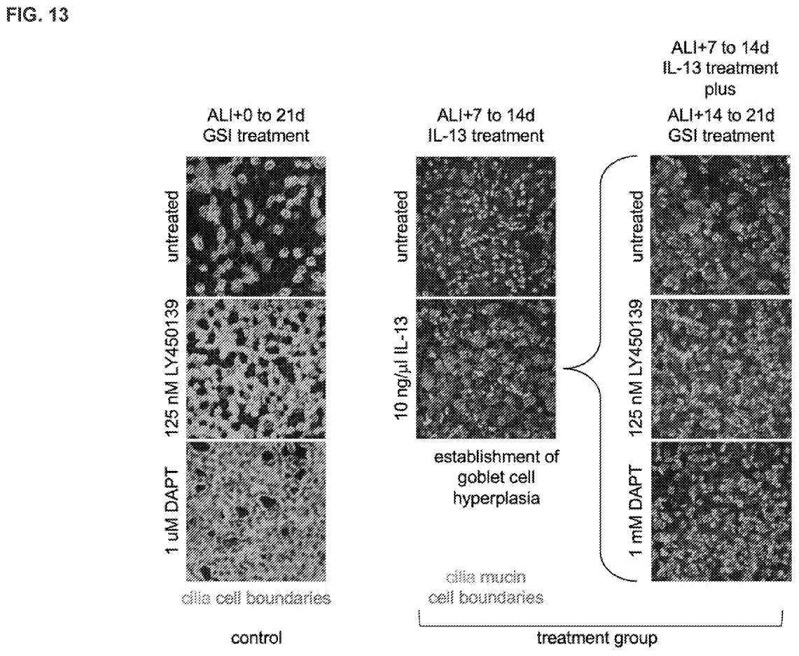

FIG. 13 shows the effect of semagacestat and DAFT treatment in an ALI model of chronic inflammation. HNEC cultures from a CF patient were treated with IL-13 from ALI+7 to 14 to induce inflammation. DAFT and semagacestat increase the percentage off MCCs in controls (left). IL-13 treatment increases the percentage of mucin positive secretory cells and decreases the percentage of MCCs. Subsequent DAFT or semagacestat treatment rescues cell composition, increasing the percentage of MCCs and decreasing the percentage of mucin positive secretory cells.

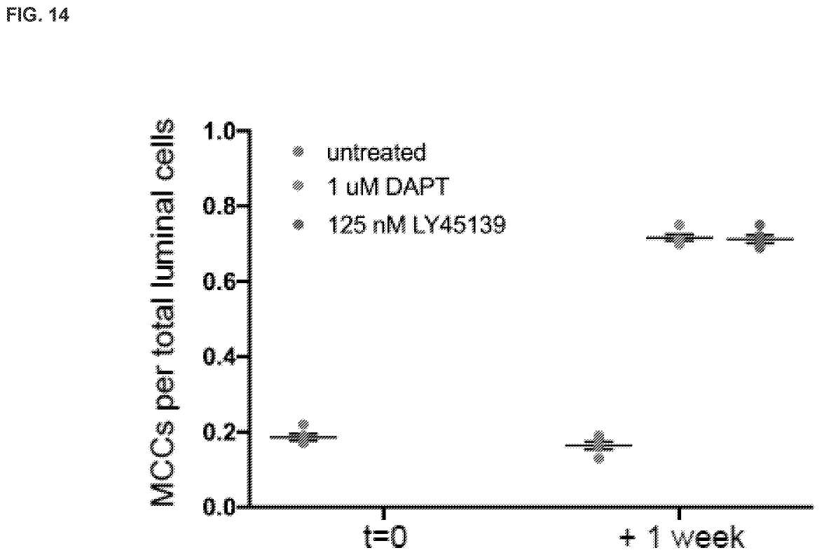

FIG. 14 shows the quantitation of MCCs per total luminal cells of the data from FIG. 13.

### Example 5: Representative Ussing Chamber Tracings

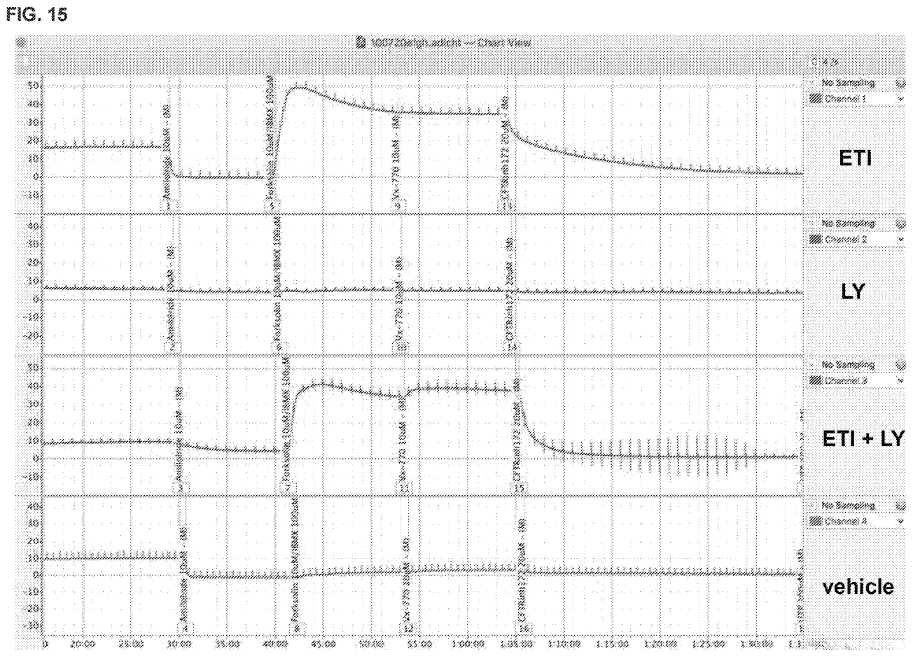

Cells grown at an air liquid interface were mounted on a holding slider and inserted on an Ussing chamber for electrophysiological short circuit current (Isc) measurements. Solutions in the serosal and mucosal bath were prepared so that a chloride gradient was established between both sides. After stable baseline current recordings were obtained, agonists were added in the following order: amiloride (10 μM) to block sodium channel activity, forskolin (10 μM) to stimulate CFTR, Ivacaftor (10 μM) to potentiate CFTR activity, and CFTRᵢₙₕ-172 (20 μM) to block CFTR current. For each agonist signals were monitored until a plateau in current was noted before adding the next agonist. The delta-Isc in response to CFTRᵢₙₕ-172 was used as our main read out for CFTR-mediated chloride transport. Results are shown in FIG. 15.

### Example 6: Electrophysiological Assay for CFTR Activity

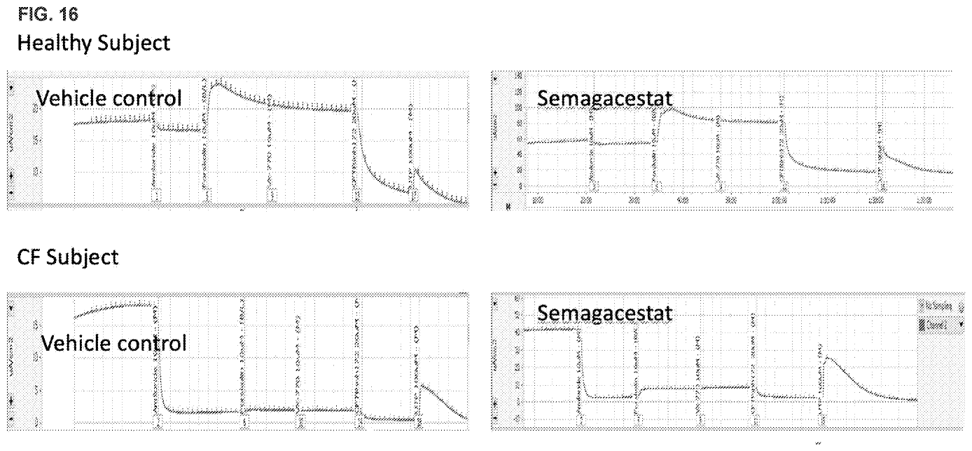

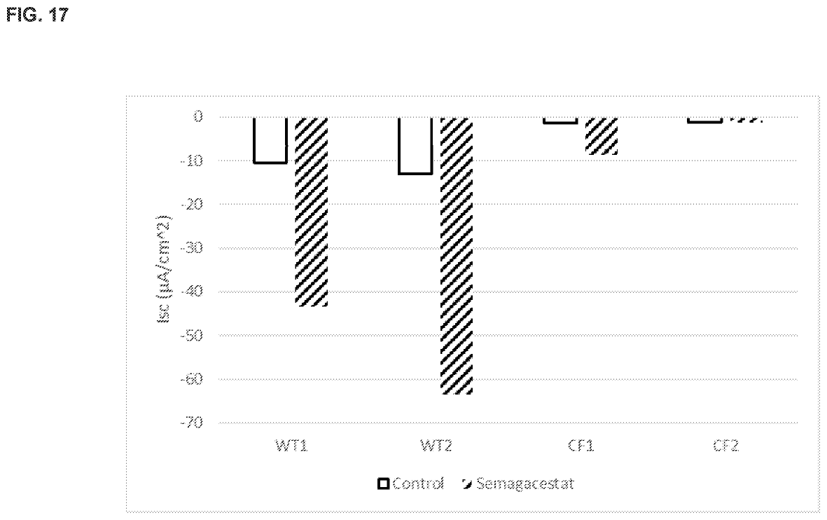

To assess the effect of GSIs (DAPT, Semagacestat) on CFTR function in cultured epithelia, HNECs from CF patients and non-CF controls were collected and grown at air-liquid interface to maturity (+21d) according to Vladar et al. Cultures were then treated with DAPT, Semagacestat, the CFTR modulator Lumacaftor or vehicle control added to basal media 3× per week for 2 weeks. Filter inserts were then assessed for short circuit current (Isc) against a chloride gradient in Ussing chambers to assess CFTR activity. FIG. 16 shows representative tracings. FIG. 17 quantifies CFTR channel activity as assessed by inhibition of current after addition of CFTRᵢₙₕ 172 for two wild-type control cultures and two CF patient-derived cultures (genotypes: rare/rare=W 1282X [class I]/I1234V [class II] and F508Δ/F508Δ). Note that in all cases, CFTR currents in semagacestat treated cultures are equal to or greater than in control conditions.

### Example 7: Mucus Production in Human CF Cells

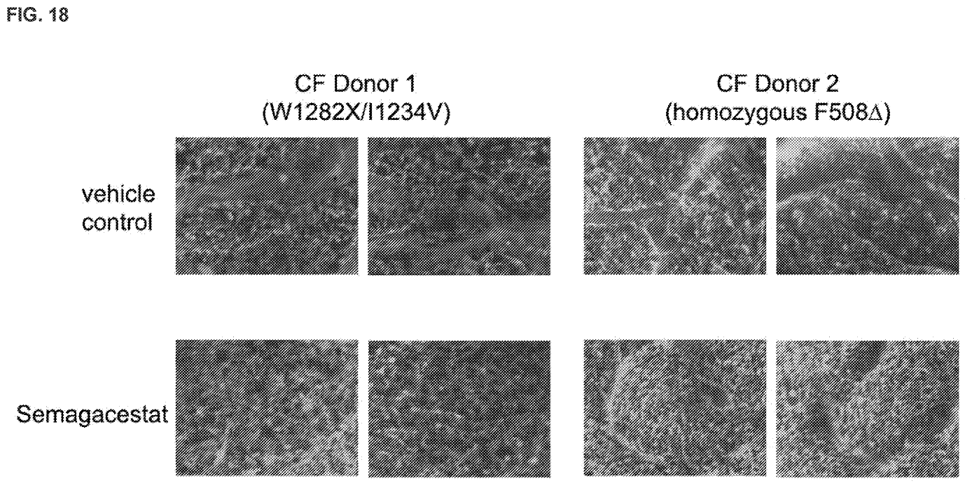

In FIG. 18, duplicate cultures from the experiment in Example 6 were PFA fixed and stained for Muc5AC (red; mucus) and Acetylated tubulin (green; MCCs). Note the thick ropes of mucus (red) in vehicle control treated CF cultures that were resistant to washing. Semagacestat treated cultures revealed much less mucus without ropes. The effect was observed in homozygous F508Δ and W1282X/11234V (a rare modulator-responsive genotype) cells under treatment with the CFTR modulator combination of lumacaftor and ivacaftor.

### Example 8: Combination of Semagacestat and CFTR Modulator

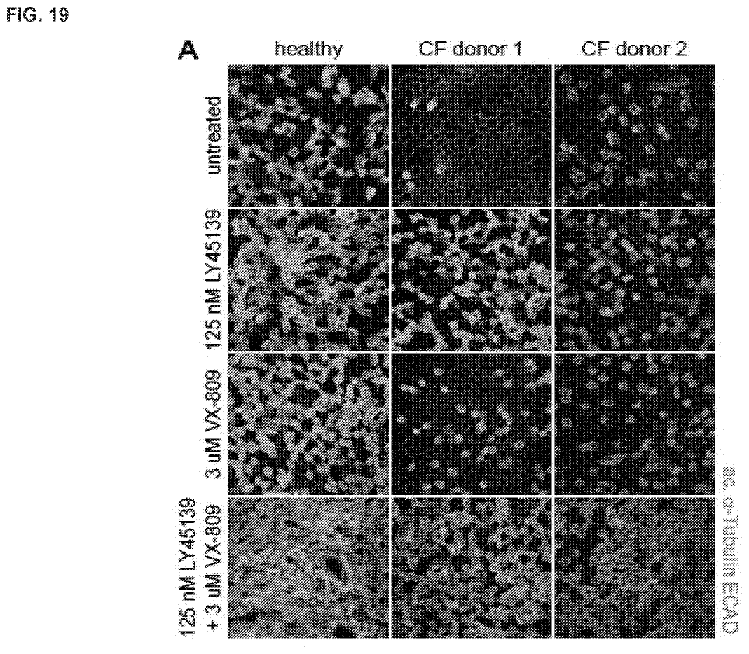

To assess the effects of combining semagacestat and CFTR modulator treatment, wild-type control and CF (F508AIF5080) HNEC cultures were grown to maturity with or without semagacestat from ALI+0-21d and cultures were treated with or without Lumacaftor (VX-809) from ALI+19-21d and Ivacaftor (VX-770) for ten minutes prior to fixation. PFA fixed membranes were then stained for Acetylated tubulin (green; MCCs) and E-Cadherin (red). Results are shown in FIG. 19. Note that combination treated cultures differentiated MCCs as well as or better than cultures treated with semagacestat alone.

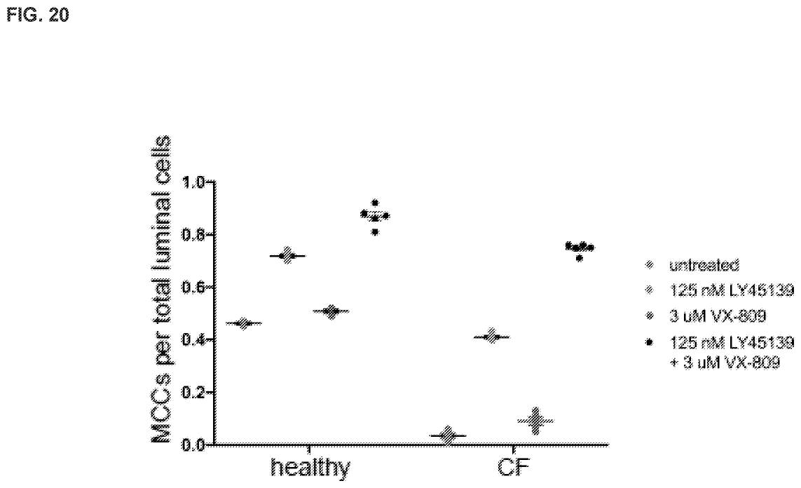

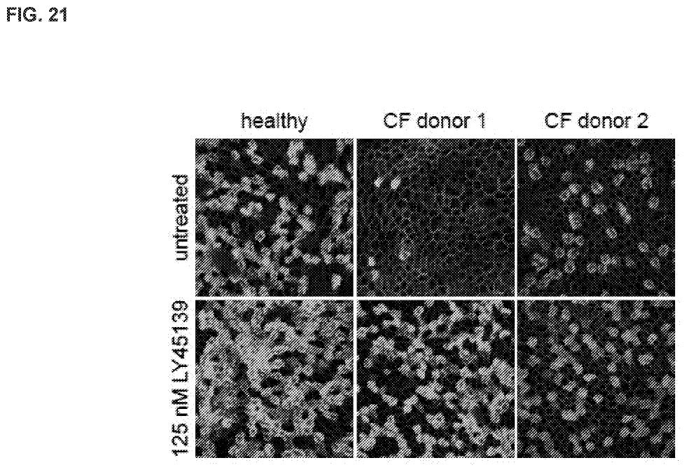

FIG. 20 shows the quantitation of data for healthy patient and CF donor 1 in FIG. 19. In some cases, such as this one, the semagacestat-induced increase in MCC/total cell ratio is further increased by Lumacaftor. Although this was not consistently seen and is not statistically significant among all cultures we have quantified, we can conclude that no significant decrease in semagacestat-induced MCC response was seen. CF patients not eligible for Trikafta respond to semagacestat alone similarly to those on corrector therapy (not shown).

### Example 9: GSI Treatment Induces Multiciliated Cell Formation in Cystic Fibrosis Epithelia

Primary healthy and cystic fibrosis airway epithelial cells were treated with semagacestat (LY45139) during differentiation only (ALI+0 to +21 d) and labeled at ALI+21d with anti-acetylated α-Tubulin (green) and ECAD (red) antibodies. FIG. 21 shows that LY45139 was effective in increasing MCC/total cell ratio in CF patient-derived cultures. Restoration of a healthy MCC/total cell ratio is expected to improve mucociliary clearance.

### Example 10: GSI Treatment Induces Structurally Normal Cilia in Healthy and CF Epithelia

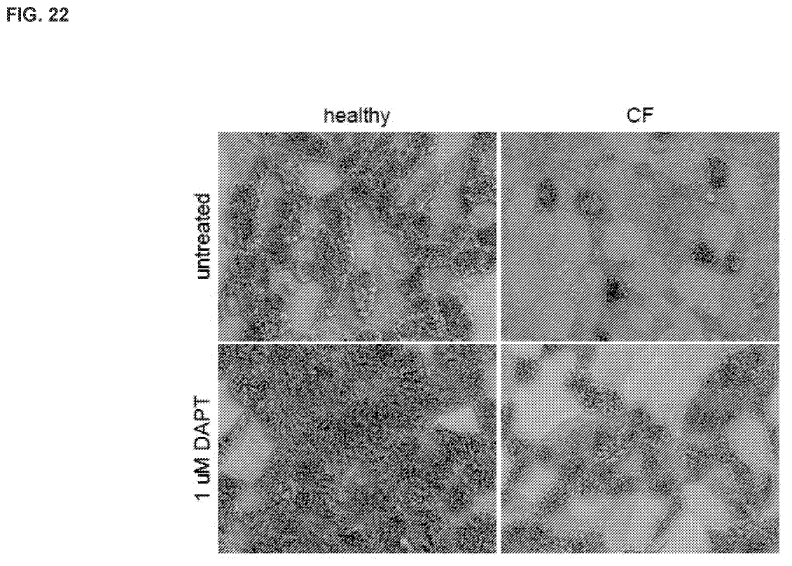

FIG. 22 shows SEM of healthy and CF primary human airway epithelial cultures, showing that multiciliated cells formed under DAPT treatment are indistinguishable from those in untreated healthy cultures.

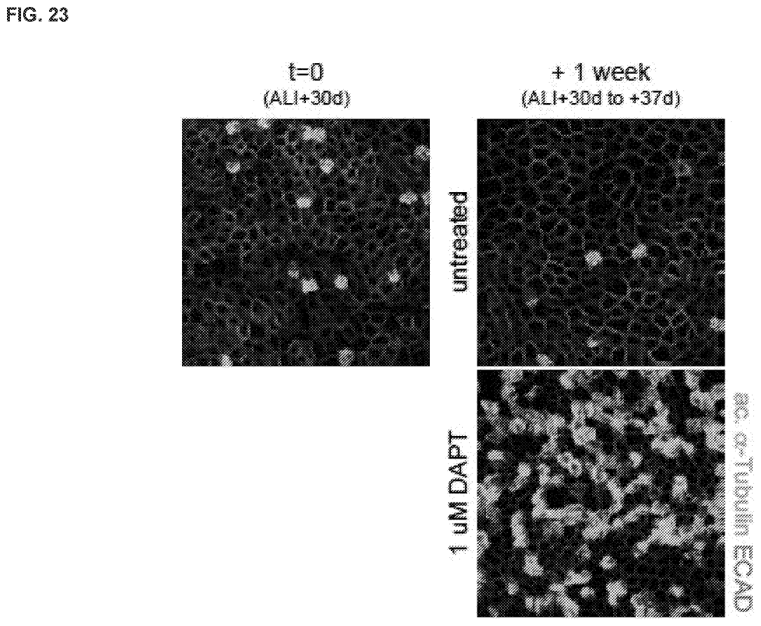

Mature (ALI+30d) primary cystic fibrosis human airway epithelial cells were treated with DAPT for one week (ALI+30 to +37d), then labeled with anti-acetylated α-Tubulin (green) and ECAD (red) antibodies. The results shown in FIG. 23 show that GSI treatment induces the formation of additional multiciliated cells in mature cystic fibrosis cultures, while untreated cultures do not differentiate any more multiciliated cells.

### Example 11: Multiciliated Cell Formation is Induced by a Variety of GSIs

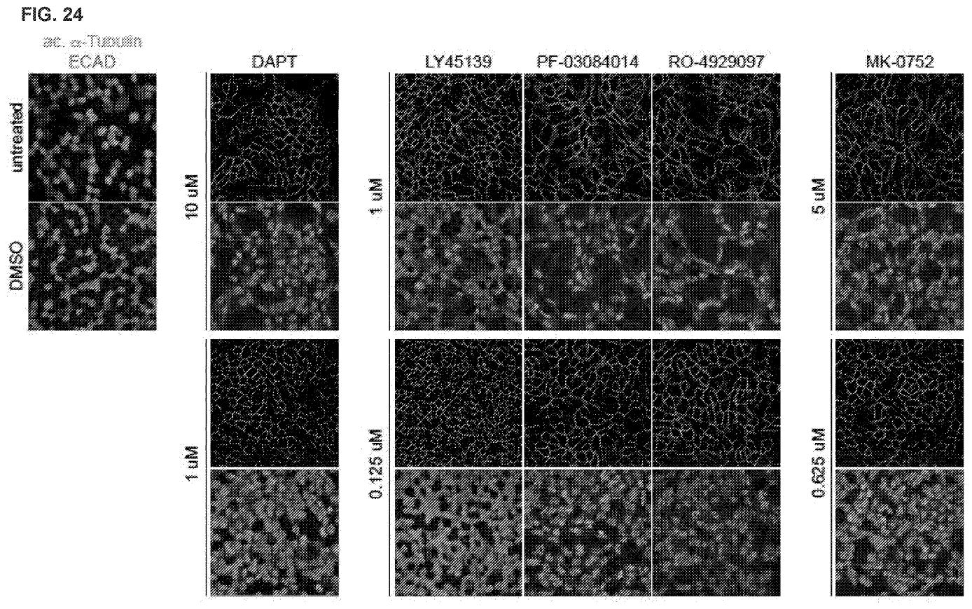

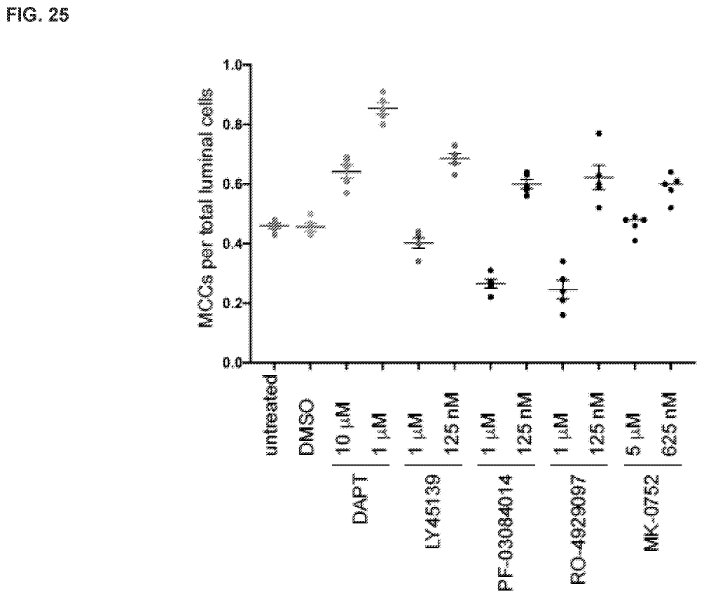

Primary human airway epithelial cells were treated during differentiation (ALI+0 to +21d) with DAFT and high and low concentrations of the GSIs LY45139, PF-03084014, RO-4929097 and MK-0752, and labeled at ALI+21d with anti-acetylated α-Tubulin (green) and ECAD (red) antibodies to mark cilia and epithelial junctions. Results are shown in FIG. 24. At high concentrations, GSIs disrupted epithelial structure, but at the low concentration all induced multiciliated cell formation, similar to DAPT. FIG. 25 shows the quantitation of MCCs per total luminal cells of the data shown in FIG. 24.

### Example 12: CFTR Current is not Diminished by GSI Treatment

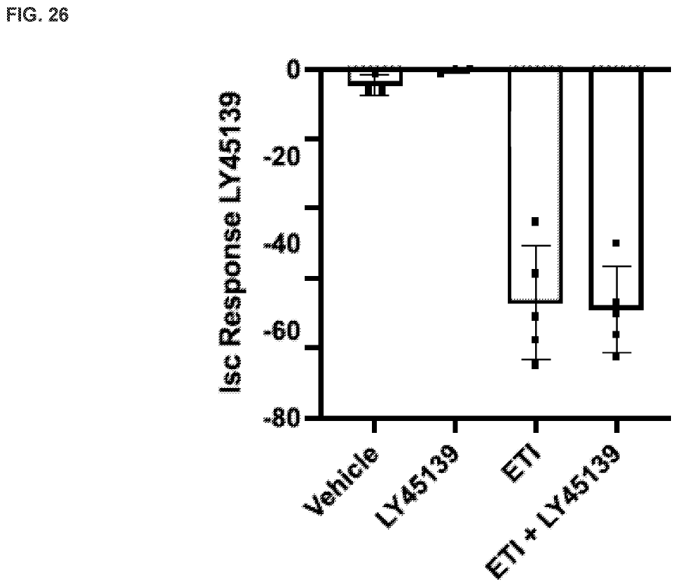

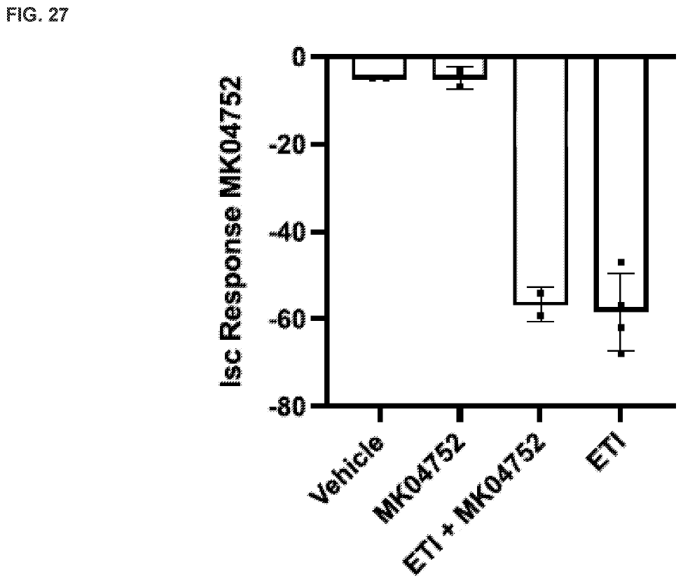

We directly measured CFTR activity in cultures from CF patients treated with GSI with or without well-established effective in vitro doses of Trikafta (ElexcaftortTezacaftor/Ivacaftor or “ETI”). Veit, G., et al., JCI (2020) 10.1172/jci.insiglx.139983. Measurements were performed in Ussing chambers. Neither LY45139 (FIG. 26) nor MK04752 (FIG. 27) impaired the CFTR current induced by ETI. In some individual experiments, there appeared to be a synergistic effect in the combination treatment: GSI alone has no effect but the combination treatment produced a greater response than ETI alone. This effect was not consistently seen, but raised the possibility that synergy might be observed with lower ETI doses.

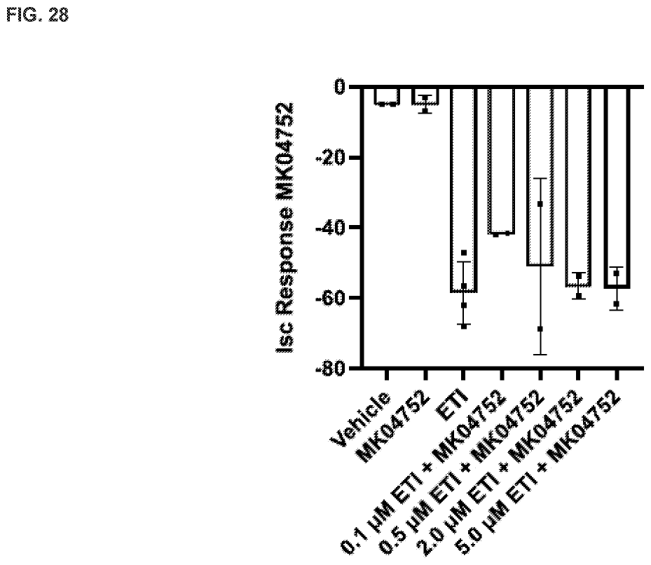

To examine this further, we tested MK04752 in combination with varying doses of ETI (FIG. 28). The results show that suboptimal doses of ETI appear to be potentiated by MK04752, a result that indicates synergy.

### Example 13: BSE Treatment has No Impact on Ionocyte Formation

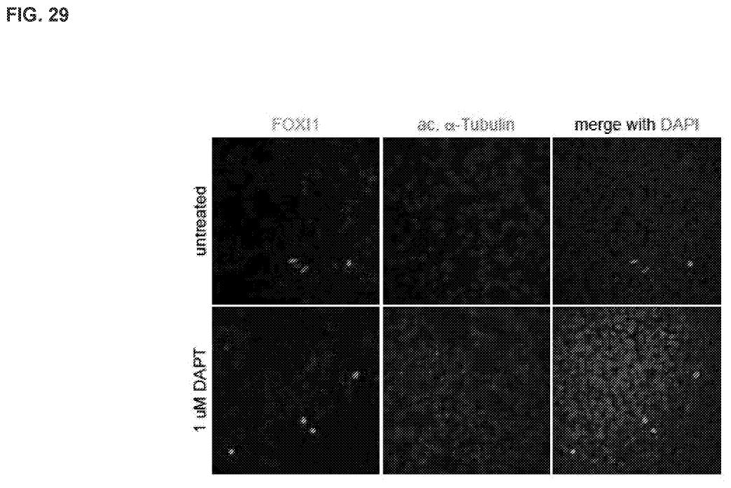

Since ionocytes are of particular interest due to prior assertions about CFTR expression, in addition to measuring current, we assayed ionocyte prevalence with GSI treatment. Primary healthy airway epithelial cells were treated with DAPT during differentiation only (ALI+0 to +21d) and labeled at ALI+21d with anti-FOXI1 (green; an ionocyte specific marker), and acetylated α-Tubulin (red) antibodies and stained with DAPI (blue) to mark nuclei. FIG. 29 shows that both untreated and DAPT treated cultures contained a similar small number of FOXI1 positive nuclei, indicative of ionocytes. Therefore, the prior suggestion that ionocyte numbers would decrease with Notch inhibition appears not to be correct.

### Example 14: Effect of Low Concentrations of the GSI MK-0752 and the CFTR Modulator Elexacaftor on

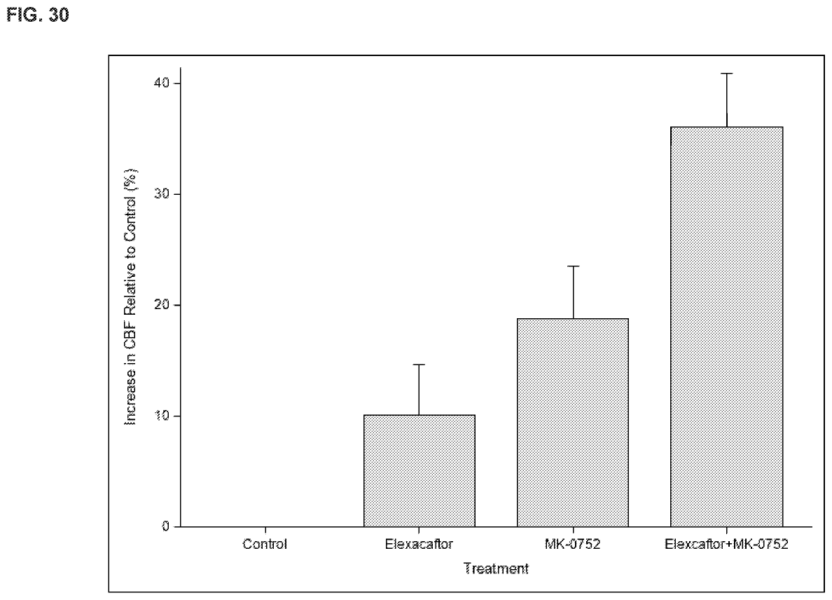

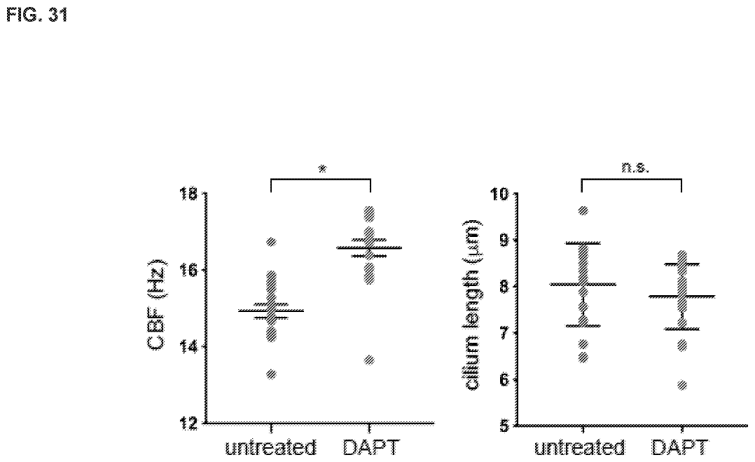

Primary nasal epithelial cells from two CF donors (F508del homozygotes) were grown in filter inserts as in previous experiments to full differentiation. We evaluated whether lower doses of both MK-0752 and Elexacftor than doses used in the previous experiments could elicit a positive response in CBF as evidence for synergy between the two drugs. During differentiation, cultures received treatment with vehicle control or the GSI MK-0752 at 125 nM, or triple combination modulator combination with Elexacaftor at 100 nM, or both treatments combined. Once cells reached maturity, the apical surface was washed gently with PBS and then placed on an inverted microscope on a heated stage at 37° C. High speed video recording at 200× of the ciliated surface was performed to estimate the CBF in several regions. Average CBF in Hz for each condition are represented by the bars in FIG. 30. Both treatments demonstrated a significant effect in increasing the CBF (p<0.001 for both vs control). Further, the increase in CBF elicited by the combined treatments was larger than with either drug alone (p<0.005 for all comparisons) and demonstrating synergy as the increase in CBF was significantly above that expected by the simple addition of their independent effects (36% vs 28%, p=0.049).

FIG. 31 shows ciliary beat frequency (CBF) and cilium length of primary human epithelial cells treated with DAPT versus unheated controls. The results show that multiciliated cells differentiated in the presence of DAFT treatment had a modest, but significant increase in ciliary beat frequency, but showed no difference in cilium length.

### Example 15: Airway Surface Liquid (ASL) Reabsorption Characteristic of CF is Attenuated by GSI Treatment to the Same Degree as CFTR Modulator Drugs

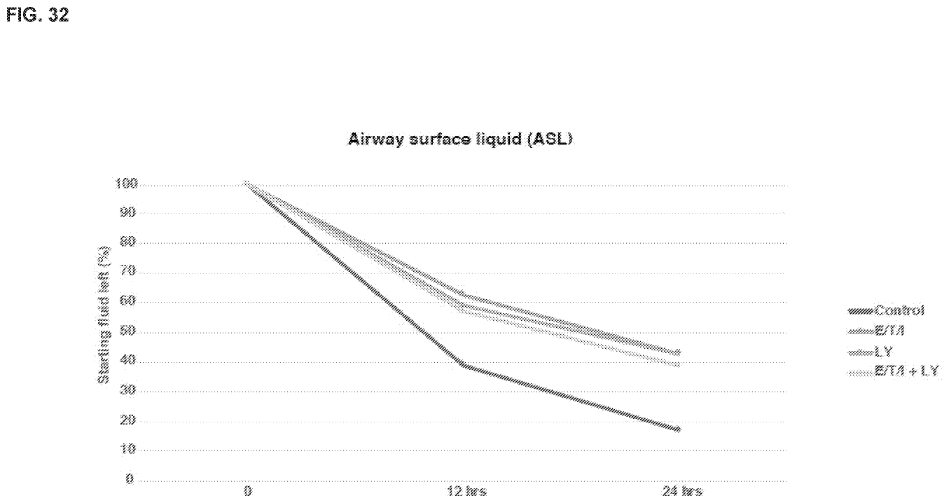

Primary nasal epithelial cells from two CF donors (F508del homozygote) were grown in filter inserts as in previous experiments to full differentiation. During differentiation they received treatment with vehicle control (blue), triple combination CFTR modulator (Elexcaftor/Tezacaftor/Ivacaftor; orange), the GSI semagacestat (LY-45139) (gray) or both treatments combined (yellow). Once they reached maturity, the apical surface was washed gently with PBS and then 30 μl of PBS were added to the surface. The filter inserts were then weighed on a precision scale at times 0, 12 and 24 hours after fluid addition. The change in weight over time was taken as a surrogate for fluid reabsorption. Results are shown in FIG. 32. Control cells showed the typical pattern of ASL reabsorption over a 48-hour period, as opposed to treated cells that demonstrated significantly decreased reabsorption (p=<0.001 vs control). Notably, all drug treatments did not significantly differ in their effect on fluid reabsorption (p>0.3 for all comparisons between drug treatments).

### Example 16: Dramatic Improvement in Mucus Transport with Combined GSI and CFTR Modulator Treatment

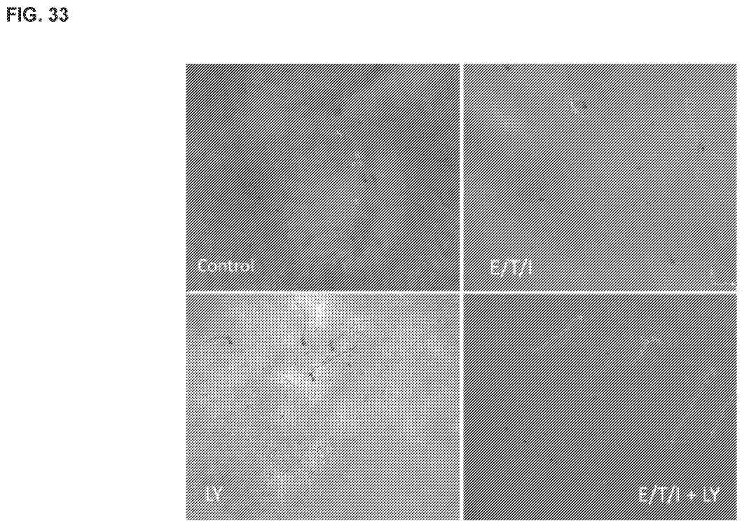

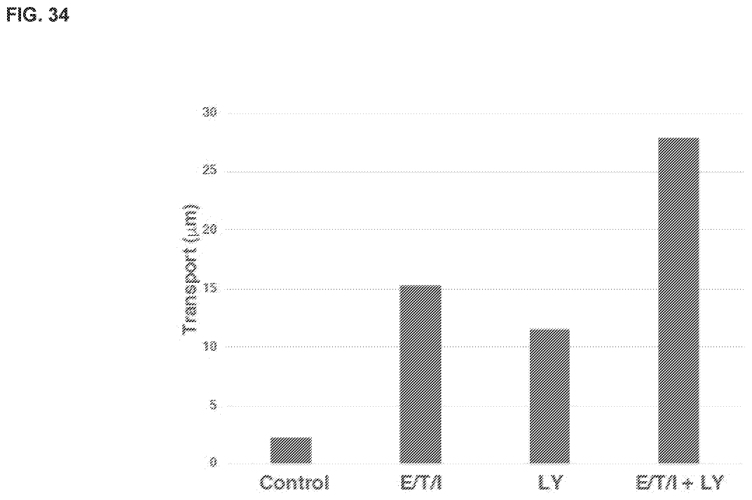

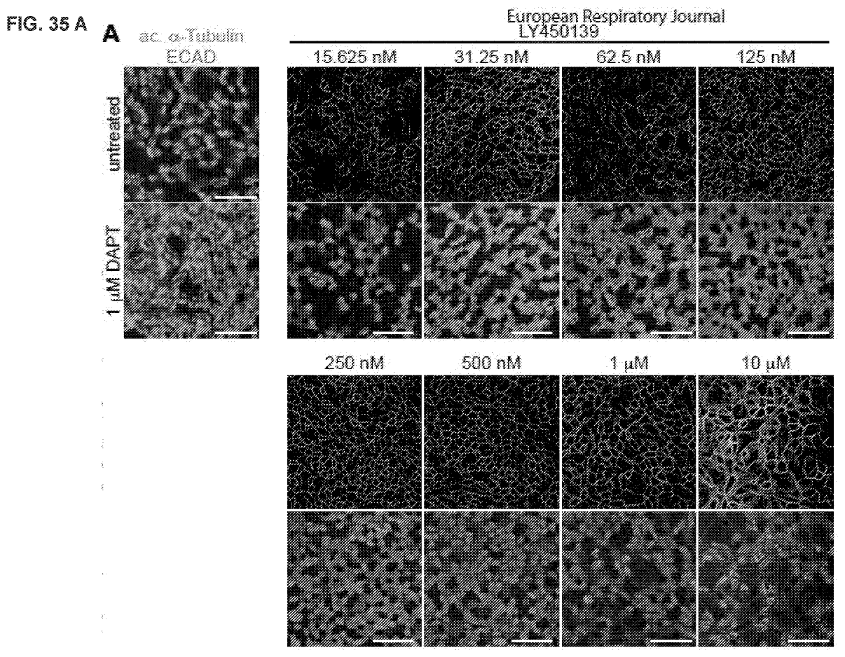

Primary nasal epithelial cells from two CF donors (F508del homozygotes) were grown in duplicate as in previous experiments to full differentiation under treatment with vehicle control, triple combination CFTR modulator (Elexcaftor/Tezacaftor/Ivacaftor), the GSI semagacestat (LY-45139) or both treatments combined. Once mature, a 20 μl suspension of 2 μm latex beads was added to the apical surface and the insert cut for placement under a microscope fitted with a high-speed video recorder. Images were then acquired at 1000 fps to track bead movement as a reflection of mucus transport by the ciliated surface and distance travelled by individual beads estimated. FIG. 33 shows representative images from each treatment. Results shown in FIG. 34 show that control cells showed little movement of beads, reflective of poor mucus transport. Significant increases in movement were observed with either ETI or semagacestat treatment (p<0.01 for either treatment vs control). Remarkable increases in transport were noted with the combination of ETI and semagacestat (p<0.05 for comparisons against either treatment alone). This can be taken as demonstrating strong enhancement effects in mucociliary transport upon combination treatment, and predicts substantial benefit to patients on CFTR modulator therapy by adding GSI treatment.

### Example 17: Selection of a Small Molecule γ-Secretase Inhibitor (GSI) to Increase the Proportion of Multiciliated Cells in Healthy Airway Epithelia by Inhibition of Notch Signaling

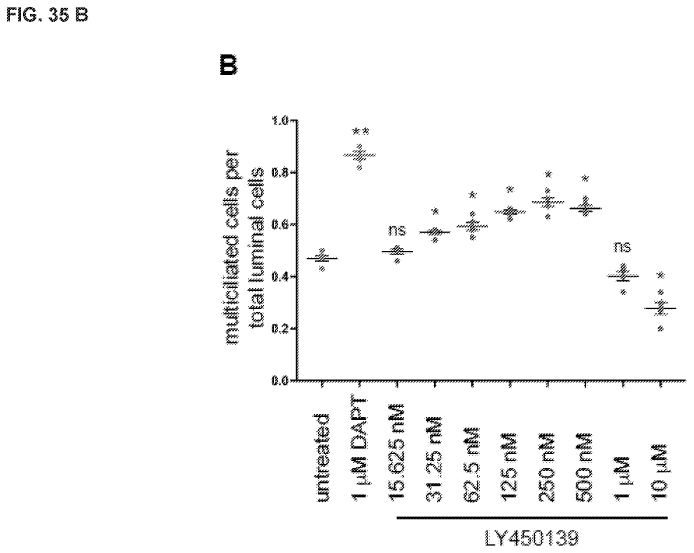

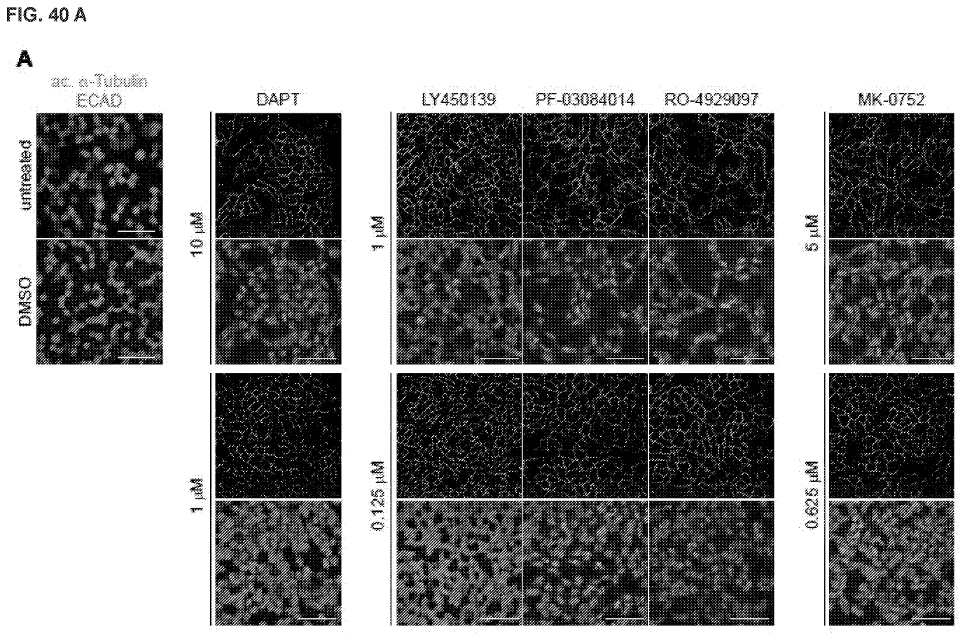

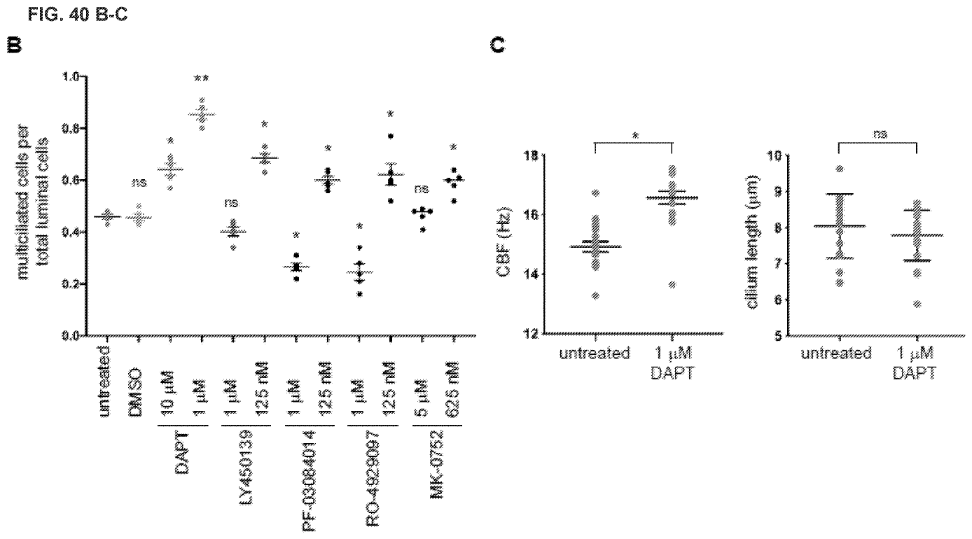

The γ-secretase inhibitor (GSI), DAPT, is known to block Notch signaling and increase the proportion of multiciliated cells in an in vitro reconstituted respiratory epithelium (23,24). While this effect may be beneficial as a therapeutic, DAPT is not suitable for human use. It was sought to identify GSIs for potential therapeutic application among small molecules previously used in clinical trials that were developed to have acceptable drug-like properties. Several candidate GSIs were selected based on literature searches (17,26) and their commercial availability from a trusted manufacturer: LY450139 (semagacestat), PF-03084014 (nirogacestat), RO-4929097 and MK-0752. Healthy human nasal epithelial cell (HNEC) primary cultures were grown at air-liquid interface (ALI) in homemade culture medium to support the consistent differentiation of multiciliated cells as approximately 45-55% of the luminal cells, similar to the in vivo airways (24) (FIG. 35A, 40A). The use of homemade culture medium is important, as cultures grown in commercially available Pneumacult medium often contain 85-95% multiciliated cells at the luminal surface (FIG. S4A). HNECs were treated with a low and high dose (based on IC50 for Notch inhibition, Selleckchem) of each compound during the entire 21 days of ALI differentiation (ALI+0 to 21d). The fraction of mature multiciliated cells was quantitated using wholemount ac. α-Tubulin antibody (marks ciliary axonemes) labeling. It was found that, like DAPT, all GSIs tested were able to induce the formation of extra multiciliated cells (FIG. 40A). DMSO control treatment had no effect on multiciliated cell number (FIG. 40A).

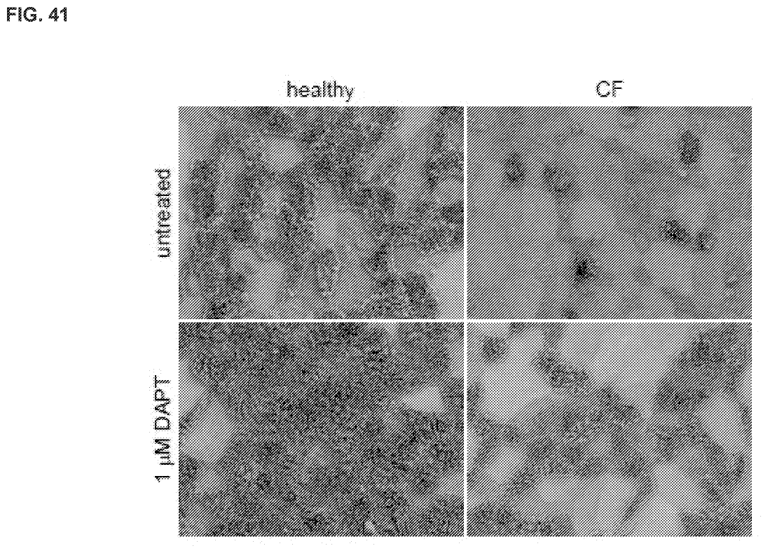

Here, LY450139 was further characterized, as of the four candidates, it is the most extensively studied GSI in both in vitro and in vivo models as well as clinical trials (28). It was determined that LY450139 treatment was able to effectively increase the proportion of multiciliated cells at as low as 31.25 nM concentration and begins to plateau at around 125 nM (FIG. 35A-B). There was a significant increase in the proportion of multiciliated cells when the cells were treated with 15.625 to 500 nM of LY450139 (FIG. 35B). There was also a significant decrease in the proportion of multiciliated when the concentration of LY450139 was increase to 10 μM (FIG. 35B). At doses in the μM range, all GSIs, including DAPT disrupted epithelial structure and multiciliated cell formation (FIG. 35, 40). It was shown that GSI treatment at doses in the nM range led to the addition of structurally and functionally normal multiciliated cells. It had no effect on ciliary length (FIG. 40C), overall morphology (FIG. 41), or the number of cilia per cell (not shown). GSI treatment slightly increased ciliary beat frequency, although it remained within the range reported for human sinonasal cilia (29) (FIG. 40C).

### Example 18: Establishing the Effective GSI Treatment Time Window and Mode of Exposure in Healthy Airway Epithelia

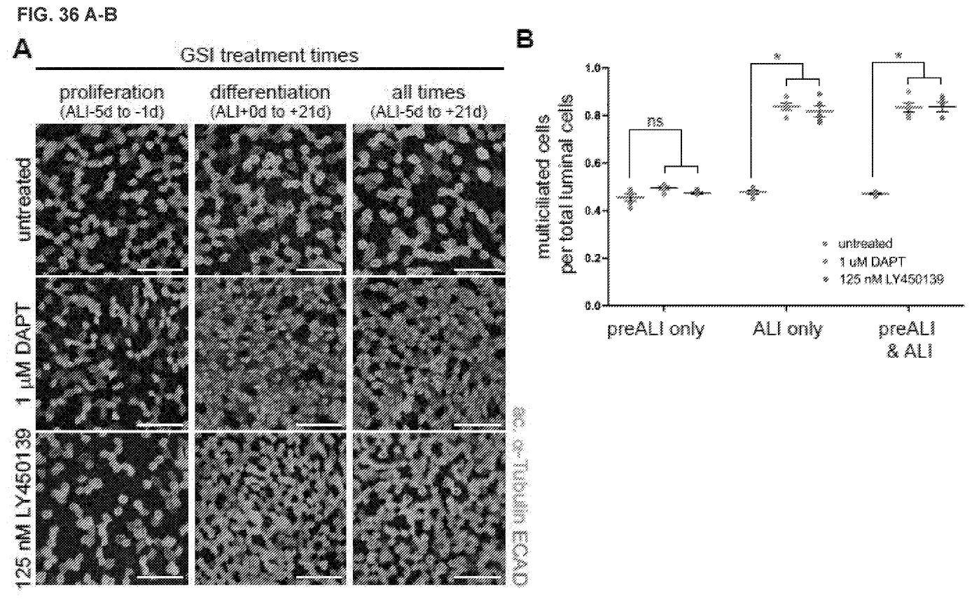

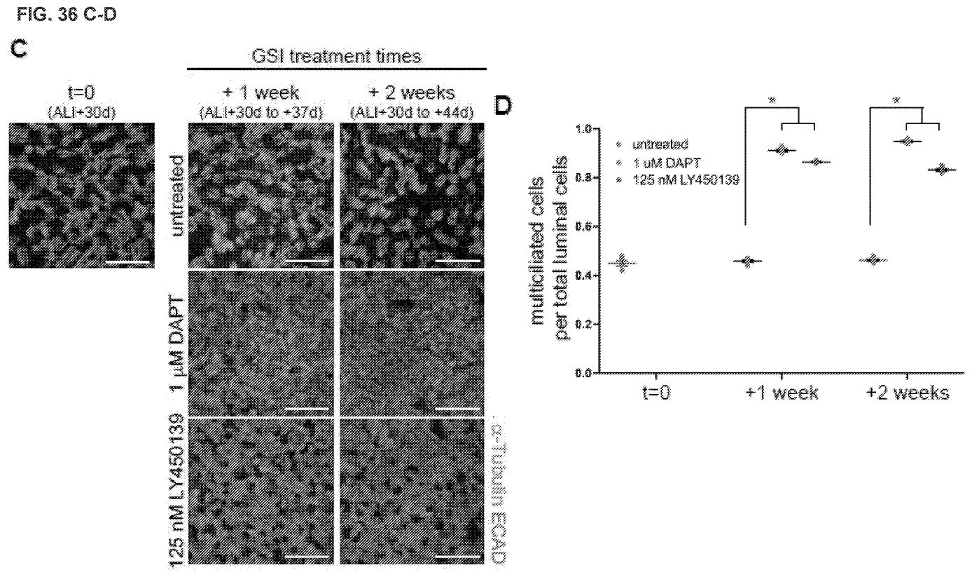

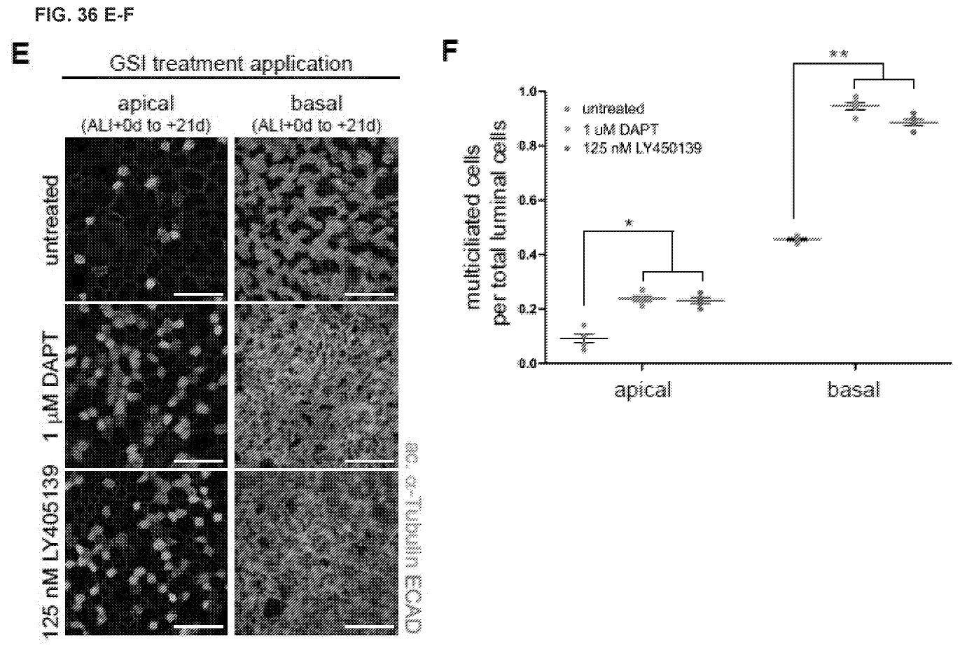

HNEC primary culture comprises an initial proliferative phase when basal stem cells establish the epithelial layer under submerged culture conditions (preALI), followed by differentiation induced, in part, by lifting to ALI (30). Thus, HNECs can be used to test GSI response in both regenerative and homeostatic settings. Notch signaling between luminal cells controls epithelial cell fate, but it is also active in basal cells and regulates their differentiation and survival (14,18,31). Thus, it was asked if the GSIs LY450139 or DAFT during the proliferative preALI phase only or during the entire culture duration (proliferative+differentiation; preALI+ALI) can induce multiciliated cell formation and if GSI treatment during stem cell proliferation has detrimental effects. PreALI, GSIs was added to both the apical and basal media, and during ALI, cultures were only treated basally. It was found that preALI only treatment was not sufficient to induce extra multiciliated cell formation. Both ALI only and continuous (preALI+ALI) treatment led to a significant increase in the proportion of multiciliated cells with no apparent disruption of epithelial composition or structure (FIG. 36A-B). This indicates that the permissive window for GSI activity is after ALI+0d and that continuous treatment does not adversely affect the induction of multiciliated cell formation.

To test if active differentiation is required or if treatment of already differentiated epithelium is sufficient to gain more multiciliated cells, GSI treatment was carried out in mature HNECs. To ensure that the cultures are fully differentiated, cultures were grown to ALI+30d. Cells were then treated with LY450139 or DAFT for one or two weeks. Both GSIs and treatment times (1 week and 2 week) were found to lead to the robust induction of multiciliated cell formation that was significantly higher than untreated conditions at the same time points (FIG. 36C-D). This indicates that GSI blockage of Notch signaling can be used to increase the fraction of multiciliated cells in both regenerating and intact epithelia.

Whereas ALI cultured HNECs are exposed to drug from the culture medium on their basal sides, it was asked if GSI treatment applied to the apical surface is sufficient to induce multiciliated cell formation. This required the long term culture of HNECs undergoing ALI differentiation under submerged conditions which has been shown to suppress differentiation (7,24). Indeed, fewer multiciliated cells were observed in submerged untreated cultures (submerged cultures were given 250 μl of apical medium, FIG. 36E-F). However, similar to ALI cultured cells that received LY450139 or DAPT basally, apical only LY450139 or DAPT treatment of submerged cultures was still able to approximately double the number of multiciliated cells which was a significant increase over untreated submerged cells (FIG. 36E-F). This suggests that LY450139 could be used to increase the proportion of multiciliated cells either when administered systemically or topically to the airway epithelial surface.

### Example 19: GSI Treatment Induces Multiciliated Cell Formation in the In Vivo Healthy Adult Mouse Airway Epithelium

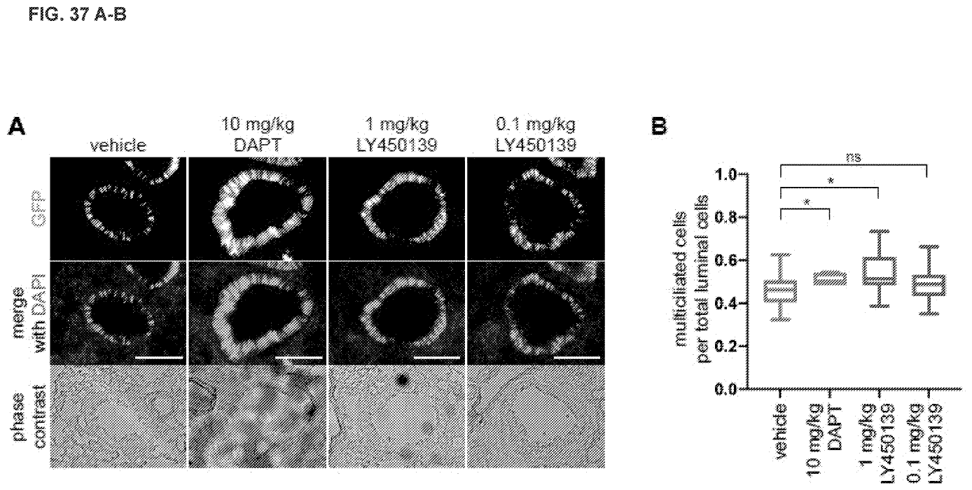

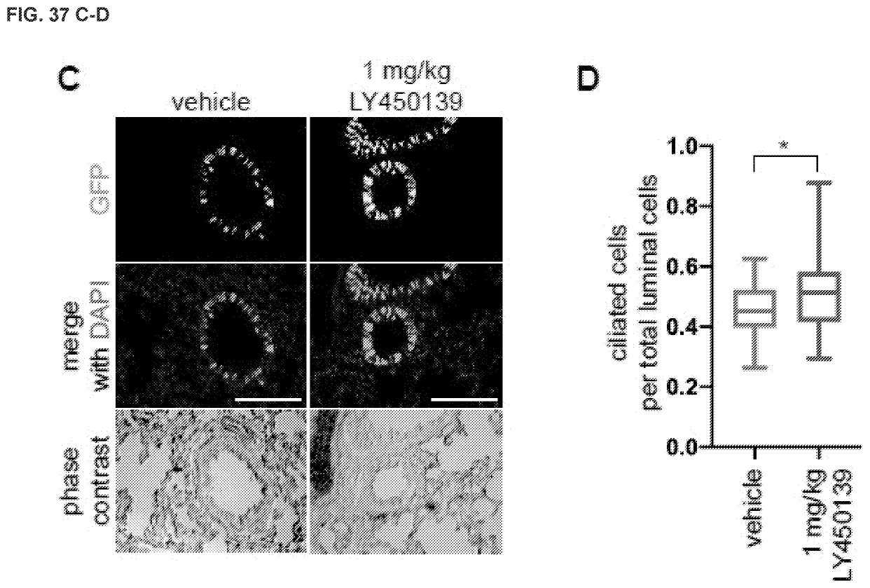

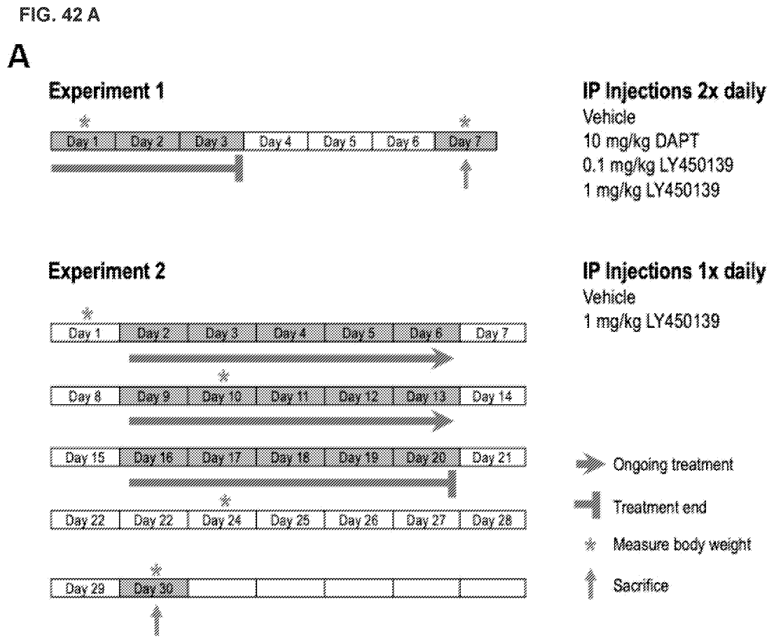

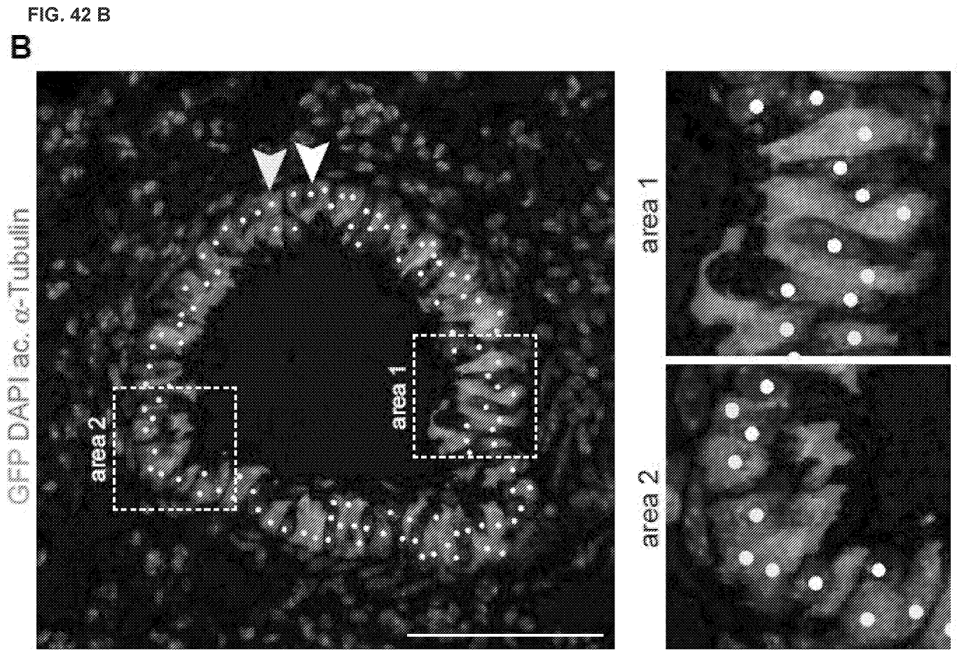

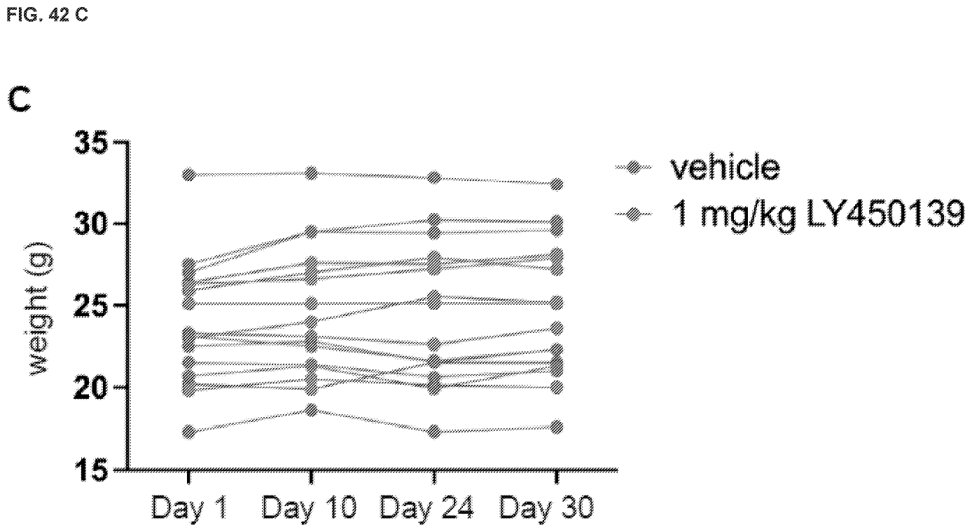

The impact of LY450139 in mouse models of neurodegeneration has been studied extensively (27,28). In these models, LY450139 were administered systemically for weeks to months at a time at up to 100 mg/kg. It was sought to determine if a substantially lower dose administered for a shorter duration would be sufficient to induce an increase in the proportion of multiciliated cells in the intact airway epithelium of healthy adult mice. Mice were first treated with 1 or 0.1 mg/kg of LY450139, 10 mg/kg DAPT or vehicle control twice daily for three consecutive days via intraperitoneal injection and then assessed on day 7 (FIG. 42A). To quantitate multiciliated cells, treatments were carried out in Foxj1/EGFP transgenic mice that expresses cytoplasmic GFP under the multiciliated cell specific Foxj1 promoter (32). Multiciliated cell number was quantitated in airways of similar size in lung tissue sections. Cells were identified by DAPI nuclear staining and were counted as multiciliated if the DAPI staining overlapped with GFP signal (FIG. 42B). It was found that 1 mg/kg LY450139 or 10 mg/kg DAPT lead to a modest, but statistically significant increase in the number of multiciliated cells, while 0.1 mg/kg LY450139 showed a trend towards more multiciliated cells that did not reach statistical significance (FIG. 37A-B). Treatment was repeated with 1 mg/kg LY450139 or vehicle control using once daily injection for five consecutive days per week for three weeks, which also induced an increase in the proportion of multiciliated cells (FIG. 42A, 37C-D). Importantly, GSI treated mice did not exhibit weight loss (FIG. 42C), or any other adverse event, indicating that the treatment regimen was well-tolerated.

### Example 20: GSI Treatment Restores Multiciliated Cell Abundance in Airway Epithelia Depleted of Multiciliated Cells Due to Chronic Inflammatory Remodeling

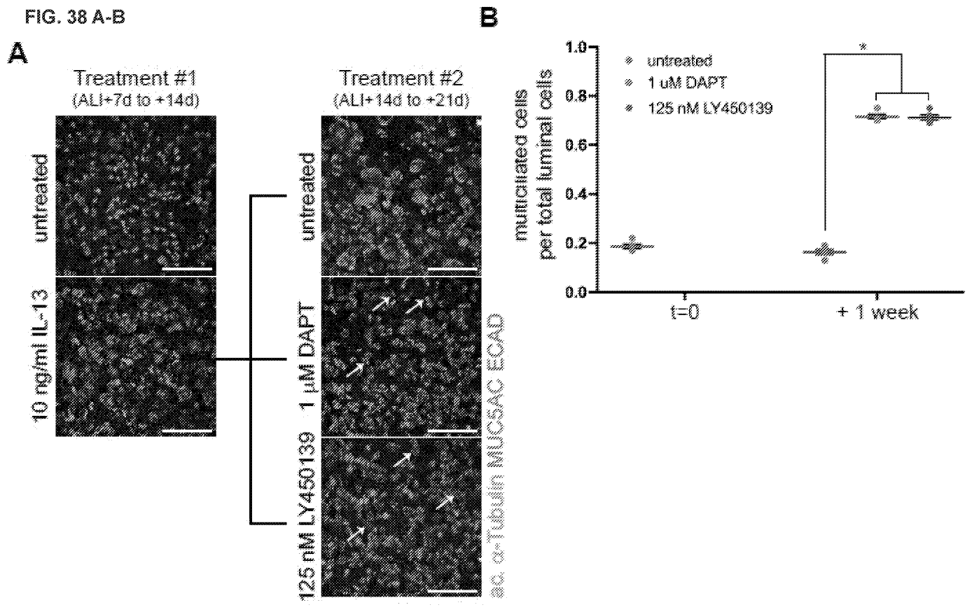

Thus far it has been demonstrated that blocking Notch signaling by GSI treatment can induce multiciliated cell formation in healthy in vitro and in vivo airway epithelia. To test whether GSI treatment has the ability to restore multiciliated cells in remodeled epithelia with fewer than normal multiciliated cells, healthy donor HNECs were treated with the IL-13 pro-inflammatory cytokine to model mucous metaplasia (7,24). IL-13 treatment from ALI+7 to 14d of culture led to the formation of excess mucus secretory cells, identified by MUCSAC labeling, at the expense of multiciliated cells (FIG. 38A). At ALI+14d, the cytokine was withdrawn, and cultures were treated for one week with LY450139, DAPT or DMSO control. Control cultures continued to exhibit mucous metaplasia with a reduced fraction of multiciliated cells (FIG. 38A-B). However, GSI treated HNECs significantly increased their proportion of multiciliated cell numbers relative to untreated conditions. This was accompanied by a reduction in the proportion of mucous cells, suggesting that GSI treatment not only induces multiciliated cell differentiation, but also relieves mucous cell hyperplasia. The presence of MUC5AC and ac. β-Tubulin double positive cells, indicating an intermediate phenotype, suggests that this occurred, at least in part, through transdifferentiation of mucous to multiciliated cells upon inhibition of Notch signaling (FIG. 38A).

GSI treatment restores multiciliated cell abundance in cystic fibrosis airway epithelia and does not interfere with CFTR correction by highly effective modulator therapy (HEMT).

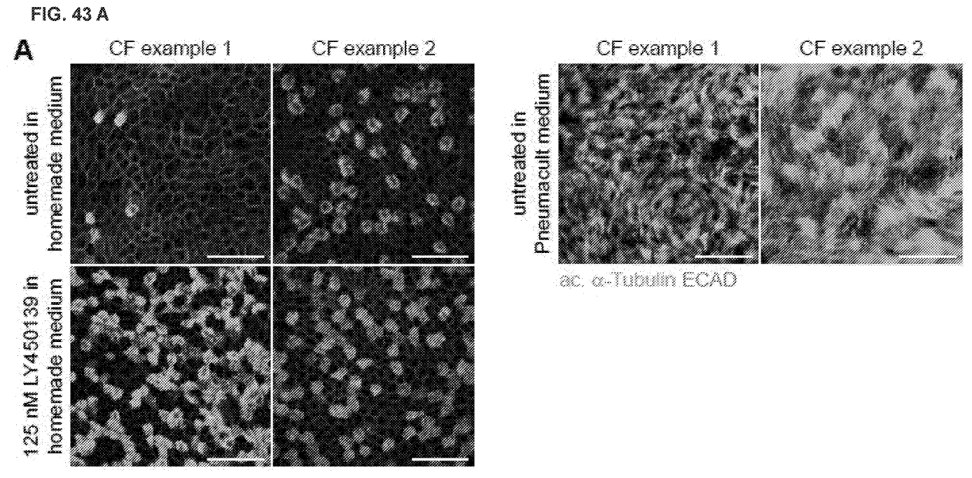

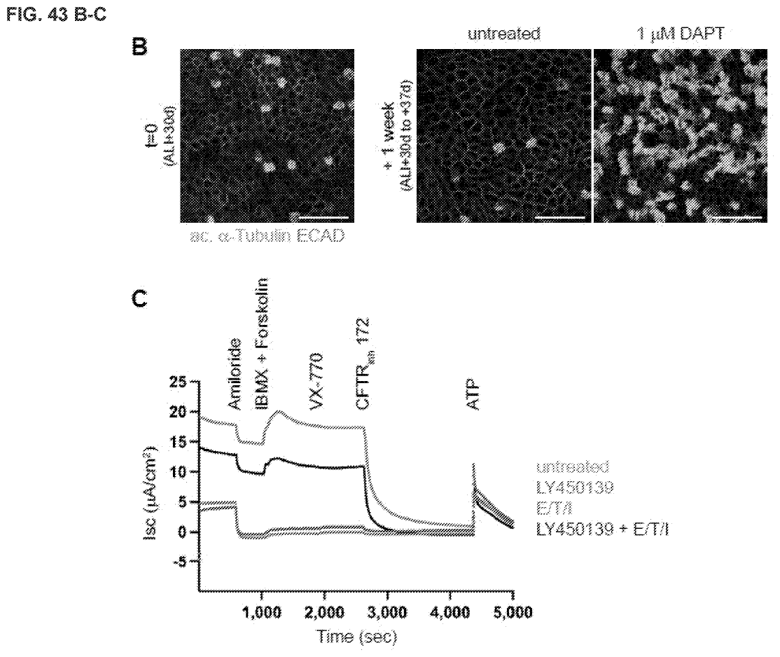

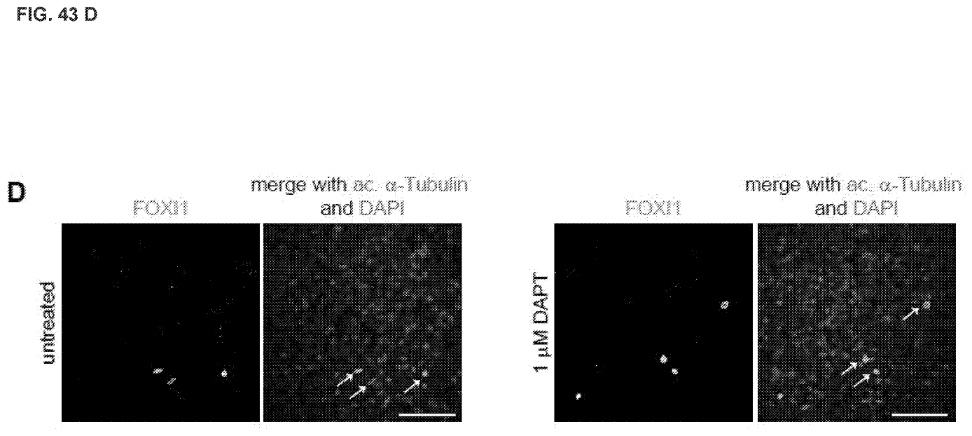

It was previously showed that CF HNECs cultured from unpassaged or early passage sinonasal basal cells in homemade medium model the reduced multiciliated cell numbers focally exhibited by explanted donor tissue (24) (FIG. 43A). In homemade medium, it was also showed that DAFT treatment during differentiation increases the multiciliated cell proportion in CF cultures to that of healthy HNECs (24). Here, it was shown that LY450139 leads to a similarly normalized proportion of multiciliated cells in CF HNECs (FIG. 43A). As observed in healthy HNECs, GSI-induced multiciliated cells in CF HNECs are structurally normal, and GSI treatment in both differentiating and mature CF cultures can mitigate multiciliated cell loss (FIG. 41, 43A-B).

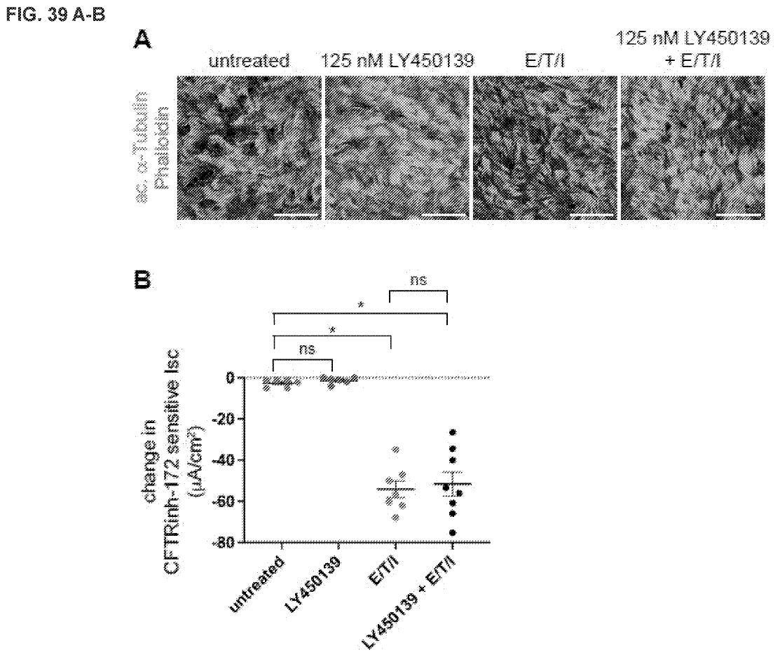

Approximately 90% of people with CF (pwCF) in the U.S. carry at least one mutant CFTR allele that is compatible with highly effective modulator therapy (HEMT), a triple combination of the small molecules VX-445, VX-659 and VX-770 (elexacaftor/tezacaftor/ivacaftor or E/T/I) (33). The majority of pwCF are receiving E/T/I, which has led to substantial clinical improvements. Thus, it is critical that administration of a GSI as a potential adjunct therapy does not interfere with the efficacy of E/T/I. To measure a potential impact on correction of CFTR function by E/T/I, CF HNECs from 4 donors (Table 1) were differentiated with and without LY450139 and E F/I (FIG. 39A). Ussing chamber analysis revealed effective correction of CFTR function by E/T/I with or without LY450139 which displayed a significant change in electrophysical short circuit current that was not observed in the untreated or LY450139 treatment alone (FIG. 39B, 43C). It was concluded that the presence of LY450139 does not interfere with E/T/I activity. It is noted that because the Ussing chamber assay has been optimized using the commercially available Pneumacult medium (FIG. 38A), the effect of LY450139 treatment on cellular composition is masked (FIG. 39A). Furthermore, it was shown that in contrast to published reports, GSI treatment in HNECs did not reduce the number of FOXI1 positive ionocytes (FIG. 43D), which are thought to serve as hotspots for CFTR activity in the airway epithelium (34). Thus, a combination of GSI treatment and E/T/I has the potential to restore both normal epithelial cell composition and function in the CF airway epithelium.

TABLE 1
CF subject characteristics.

| Subject | Sex (M/F) | Age (Years) | CFTR mutations | Studies used |
|---|---|---|---|---|
| 1 | F | 19 | F508del/F508del | Ussing chamber |
| 2 | F | 18 | F508del/F508del | Ussing chamber |
| 3 | M | 10 | F508del/F508del | Ussing chamber |
| 4 | F | 19 | F508del/W282X | Ussing chamber |
| 5 | F | 18 | F508del/F508del | Microscopy |
| 6 | M | 23 | F508del/F508del | Microscopy |
| 7 | F | 31 | F508del/F508del | Microscopy |

### DISCUSSION

This study serves as proof of principle for the therapeutic targeting of multiciliated cell loss in chronic airway diseases by pharmaceutical grade γ-secretase inhibitors (GSIs) that block the Notch signaling pathway. It was previously demonstrated that DAFT, a GSI tool compound increases multiciliated cell numbers in healthy, CF and CRS primary airway epithelial cell cultures (24). Here it was shown that low dose LY450139, a GSI that was developed to have improved drug-like properties and has been studied extensively in human clinical trials, also increases multiciliated cells numbers in healthy in vitro and in vivo airway epithelia and restores multiciliated cell numbers in CF cultures. It was shown that LY450139 increases the proportion of multiciliated cells in a dose dependent manner, and multiciliated cells generated upon LY450139 treatment are structurally and functionally normal. The primary goal of GSI treatment in chronic airway diseases would be to restore functional multiciliated cells to optimal numbers to improve mucociliary clearance, a vital host defense mechanism. It was shown that GSI treatment can increase multiciliated cell numbers after only a few days of treatment. LY450139 was able to drive multiciliated cell formation in differentiating as well as mature, homeostatic airway epithelial cultures, suggesting that GSI treatment can restore multiciliated cell numbers in both regenerating and intact portions of the airway epithelium. Importantly, GSIs increased multiciliated cells numbers in a dose dependent manner, which suggests it might be possible to control the number of extra multiciliated cells that are generated. This is critical, as mucociliary clearance requires an optimal balance of multiciliated and secretory cells, and too many multiciliated cells could be detrimental to host defense. It was shown that LY450139 treatment during airway stem cell proliferation had no observable effect on subsequent differentiation, which suggests that it does not perturb the stem cell pool. GSIs induce multiciliated cell formation by impinging on cellular differentiation programs, so it is likely to be a disease agnostic treatment for a wide range of chronic inflammatory diseases. In COPD and asthma, where aberrant Notch activation drives mucous metaplasia (20,21), GSI treatment would directly target this pathomechanism by blocking NOTCH receptor cleavage. It was shown that GSI treatment can also relieve IL-13 driven mucous metaplasia. This likely occurs through the transdifferentiation of mucous secretory cells to multiciliated cells. Secretory club to multiciliated cell transdifferentiation has also been documented in mice lacking JAGGED ligand activity (24).

It was previously showed that restoring multiciliated cells in CF primary cultures by GSI treatment led to improved barrier capacity as measured by transepithelial electrical resistance (TEER) (24). Notch is not known to directly regulate airway epithelial cell-cell junctions, but multiciliated cells express multiple unique apical junctional components (unpublished). This raises the possibility that optimal airway epithelial barrier function may also depend on having sufficient multiciliated cells. It was also previously showed that GSI treated CF cultures have improved epithelial scratch wound regeneration (24). The mechanism for this is unclear, but the combined data suggest that GSI treatment may broadly improve the structure and function of remodeled epithelia.

Despite the mitigation of remodeling phenotypes, chronic lung diseases will certainly still require other treatments such as antibiotics, anti-inflammatories, and/or CFTR modulators (ex. E(/I) in the case of CF. Thus, GSIs as an adjuvant is proposed and not a stand-alone therapy. The majority of PwCF receive E/T/I as part of their standard of care treatment regimen (33). It was shown that in CF epithelial cultures treated with a combination of LY450139 and E/T/I, LY450139 did not interfere with the efficacy of CFTR-mediated Cl-transport correction induced by E/T/I. It was speculated that E/T/I may even lead to greater improvement of CF lung and sinus function in a structurally and functionally normalized epithelium resulting from adjunctive treatment with a GSI.

Due to their widespread and vital functions, therapeutic targeting of developmental signaling pathways such as Notch naturally carry the risk of on-target toxicity. Gastrointestinal toxicity of GSIs observed in clinical trials is thought to be related to Notch-dependent stem cell maintenance and differentiation in the gastrointestinal epithelium (35). Preclinical studies in Alzheimer's mouse models demonstrated efficacy when LY450139 was administered at 30 mg/kg daily for five months or at 100 mg/kg daily for 12 days, but not at 1 mg/kg daily for eight days 36,37. Human Phase I trials based on these and other nonclinical data were conducted at daily doses of 50 mg for 2 weeks or 40 mg for 6 weeks and at 100 mg and 140 mg daily for 14 weeks, which showed that lower doses were well-tolerated, while higher doses were not well tolerated chiefly due to GI related adverse events (27,28,38). Development as an Alzheimer's treatment was abandoned due to both lack of efficacy and unacceptable toxicities (26-28). It was shown that 1 mg/kg LY450139 administered daily for three days was sufficient to increase multiciliated cell numbers in mouse airways without any apparent toxicity. Chronic administration (once daily for five consecutive days per week for three weeks) of this dose was also efficacious and well tolerated. Based on these data, it was predicted that efficacy at tolerated exposures may be achieved in humans with lower amounts of LY450139 dosed for shorter duration. Further, the data showing that apical treatment with LY450139 was just as efficacious as basal treatment in increasing multiciliated cells suggests that topical administration of LY450139 could be developed to potentially avoid systemic effects.

GSIs represent only one therapeutic option to block Notch signaling. Anti-NOTCH function blocking antibodies are currently in clinical trials for solid tumors (26). A combination of anti-JAGGED and JAGGED2 antibodies has been shown to promote multiciliated cell formation in airway epithelial cells (39). Finally, inhibitors have been developed to target downstream signaling events, including IMR-1, which prevents the recruitment of Mastermind-like 1 to chromatin to block Notch target gene expression (40).

Finally, it is important to note that the molecular mechanism through which GSI treatment increases multiciliated cell numbers remains unclear. Mechanisms downstream of Notch ligand presentation are poorly understood in any system, although there is evidence that ligand-receptor complexes are internalized by the signal sending cell after receptor cleavage, then degraded (41). Internalization of this ligand-receptor complex may lead to activation of early ciliogenesis regulators, however the increase in multiciliated cells when cleavage is blocked by GSIs argues against this. It is also possible that multiciliated cell fate acquisition is the default pathway in the airway epithelium unless Notch signaling is activated. This highlights a need for continued rigorous basic biology investigation of airway epithelial cell fate decisions to better understand this critical, potentially therapeutically important regulatory mechanism.

### Methods

Primary human nasal epithelial cell (HNEC) cultures. Primary human nasal epithelial cells were obtained by brush biopsy of the inferior turbinates with informed consent from subjects recruited via the Cystic Fibrosis Center at Stanford University (Human Subjects Protocol No. 42710). Primary air-liquid interface (ALI) culture of nasal epithelial cells from passage 0 (P0) or P1 basal stem cells was carried out as previously described (24,30). Briefly, cells are removed from cytobrushes with gentle enzymatic digestion. P0 HNEC cultures are initiated by seeding freshly isolated cells onto Collagen I coated Transwell filters. P1 HNEC cultures are initiated from basal cells that were first expanded on Collagen I coated plastic dishes in proliferation medium supplemented with Y-27632 (ROCK inhibitor, 10 μM), DMH-1 (BMP inhibitor, 1 μM), A-83-01, (TGF-β inhibitor, 1 μM), and CHIR99021 (WNT agonist, 1 μM)(42), all from Selleckchem. HNECs seeded onto Transwells are initially cultured submerged in proliferation medium until confluency, then lifted to ALI (considered as ALI+0d of culture) by supplying differentiation medium only from the bottom compartment. HNECs are considered mature at ALI+21d of culture. Healthy and CF HNECs used in the study were cultured in homemade media (24). CF HNECs used for the Ussing chamber measurements only were cultured in Pneumacult Ex+(proliferation) and Pneumacult ALI (differentiation) media (Stemcell). GSI compounds (DAPT, LY450139, PF-03084014, RO-4929097 and MK-0752) were obtained from Selleckchem and reconstituted in DMSO then diluted in culture medium (HNEC treatment) or saline (mouse IP injection). Healthy HNECs were treated with 10 ng/ml recombinant human IL-13 (RnD Systems).

Wholemount immunofluorescence and scanning electron microscopy of HNEC cultures. For wholemount immunofluorescence, HBECs were fixed in −20° C. methanol or 4% paraformaldehyde for 10 min as previously described (43). Transwell filters were cut out of the plastic supports and placed in a humidity chamber for staining. Samples were blocked in 10% normal horse serum and 0.1% Triton X-100 in PBS and incubated with primary antibodies for 1-2 h, then with Alexa dye conjugated secondary antibodies (Thermo Fisher) for 30 min at room temperature. Filters were mounted in Mowiol mounting medium containing 2% N-propyl gallate (Sigma). Samples were imaged with a Leica SP8 confocal microscope. For antibodies and fixation conditions, see Table 2. For SEM, Transwell filters were fixed in 2% glutaraldehyde, 4% paraformaldehyde in 0.1 M NaCacodylate buffer, pH 7.4 at 4° C. overnight. Samples were osmicated, dehydrated, dried with a Tousimis Autosamdri-815 critical point dryer. Samples were mounted luminal side up, sputter coated with 100 Å layer of Au/Pd. Images were acquired with a Hitachi S-3400N VP-SEM microscope operated at 10−15 kV, with a working distance of 7−10 mm and using secondary electron detection.

TABLE 2
Antibodies.

| Protein | Antibody | Source | Fixation |
|---|---|---|---|
| ac. α-Tubulin | ab24610 | Abcam | M or P |
| E-cadherin | 13-1900 | Thermo Fisher | M |
| MUC5AC | ab3649 | Abcam | M |
| FOXI1 | HPA071469 | Sigma | P |
| GFP | 4745-1051 | AbD Serotech | P |

M = methanol
P = paraformaldehyde

Ciliary length and ciliary beat frequency (CBF) measurement in HNEC cultures. ALI+21d HNECs were washed with warm PBS to remove mucus, then the Transwell filter was gently cut out of the plastic support. The filter was carefully folded along the center such that the folded edge exposed the mucosal surface for the evaluation of beating cilia. The folded filter was placed on a microscope slide inside a 100 μm mask, 100 μl of warm medium was added to maintain humidity, and a cover slip was immediately placed over the filter to seal the preparation. The slide was then placed on a heated stage at 37° C. and imaged with a digital video microscope fitted with a Keyence VW-9000 series high speed camera (Keyence). Images were captured at 2,000× and 500 fps on multiple areas per filter. Average CBF (Hz) was calculated from kymographs generated from the high speed videos. Ciliary length was measured from still images of the recording where the cilia were orthogonal to the apical surface.

Mouse husbandry and GSI treatment in mice. Foxj1/EGFP mice have been previously described (32,44). 10-40 week age-matched male and female Foxj1/EGFP mice were treated with LY450139 or vehicle control by intraperitoneal (IF) administration. In the first experiment, 0.1 mg/kg, or 1 mg/kg LY450139, 10 mg/kg DAPT or saline vehicle was administered twice daily for three consecutive days, then the mice were sacrificed on day 7. In the second experiment, 1 mg/kg LY450139 or saline vehicle was administered once daily for five consecutive days per week for three weeks, then the mice were sacrificed on day 30. Body weight was monitored as a surrogate measure of GI toxicity. See FIG. 42A for more info. For both studies, the lungs were cryoembedded en bloc in OCT compound (Thermo Fisher). Cryosections were formalin fixed, then labeled with anti-GFP and acetylated α-Tubulin antibodies and stained with DAPI to mark nuclei. Images were acquired using a Leica SP8 confocal microscope and analyzed by three independent blinded investigators. In airways of approximately uniform size viewed in cross section, cells were first identified by DAPI signal, and then scored either as multiciliated cells (GFP positive) or nonciliated cells (GFP negative). Ac. α-Tubulin signal was used as an additional identifying feature for multiciliated cells. See FIG. 42B for example image. All procedures involving animals were approved by the Institutional Animal Care and Use Committee of Stanford University School of Medicine in accordance with established guidelines for animal care.

CFTR activity measurement in CF HNEC cultures. CF HNECs were differentiated from ALI+0 to 21d in the presence or absence of 125 nM LY450139 with and without 3 μM elexacaftor and 3 μM tezacaftor (Selleckchem). Transwells were mounted on an Ussing chamber for electrophysiological short circuit current (Isc) measurements using standard methods45. Solutions in the serosal and mucosal bath were prepared so that a chloride gradient was established between both sides. After stable baseline current recordings were obtained, agonists were added in the following order to the apical side: amiloride (10 μM) to block sodium channel activity, forskolin and IBMX (10 μM) to stimulate CFTR through increased cAMP, ivacaftor (10 μM) to potentiate CFTR activity, and CFTRᵢₙₕ-172 (20 μM) to block CFTR current. For each agonist, signals were monitored until a plateau in current was noted before adding the next agonist. The delta-Isc in response to CFTRᵢₙₕ-172 was used as the read out for CFTR-mediated chloride transport.

### REFERENCES

1. Tiddens, H., Silverman, M. & Bush, A. The role of inflammation in airway disease: remodeling. Am J Respir Crit Care Med 162, S7-S 10 (2000). https://doi.org:10.1164/ajrccm.162.supplement_1.maic-2
2. Adam, D. et al. Cystic fibrosis airway epithelium remodelling: involvement of inflammation. J Patho 235, 408-419 (2015). https://doi.org:10.1002/path.4471
3. Van Bruaene, N. & Bachert, C. Tissue remodeling in chronic rhinosinusitis. Curr Opin Allergy Clin Immunol 11, 8−11 (2011). httpsJ/doi.org:10.1097/ACI.0b013e32834233ef
4. Hough, K. P. et al. Airway Remodeling in Asthma. Front Med (Lausanne) 7, 191 (2020). https://doi.org:10.3389/fined.2020.00191
5. Tilley, A. E., Walters, M. S., Shaykhiev, R. & Crystal, R. G. Cilia dysfunction in lung disease. Annu Rev Physiol 77,379-406(2015). https://doi.org: 10.1146/annurev-physiol-02 1014-071931
6. Chen, G. et al. IL-1beta dominates the promucin secretory cytokine profile in cystic fibrosis. J Clin Invest 129.4433-4450 (2019). https://doi.org:10.1172/JCI125669
7. Gerovac, B. J. & Fregien, N. L. IL-13 Inhibits Multicilin Expression and Ciliogenesis via Janus Kinase/Signal Transducer and Activator of Transcription Independently of Notch Cleavage. Am J Respir Cell Mot Biol 54, 554-561 (2016). https://doi.org:10.1165/rcmb.2015-022700
8. Spella, M., Lilis, I. & Stathopoulos, G. T. Shared epithelial pathways to lung repair and disease. Eur Respir Rev 26 (2017). https://doi.org-10.1183/16000617.0048-2017
9. Gudis, D., Zhao, K. Q. & Cohen, N. A. Acquired cilia dysfunction in chronic rhinosinusitis. Am J Rhino Allergy 26, 1-6 (2012). https://doi.org:10.2500/ajra.2012.26.3716
10. Erie, D. J. & Sheppard, D. The cell biology of asthma. J Cell Biol 205, 621-631 (2014). https://doi.org: 10. 1083/jcb.201401050
11. Yaghi, A., Zaman, A., Cox, G. & Dolovich, M. B. Ciliary beating is depressed in nasal cilia from chronic obstructive pulmonary disease subjects. Respir Med 106, 1139−1147 (2012). https://doi.org:10.1016/j.rmed.2012.04.001
12. Collin, A. M. et al. Loss of ciliated cells and altered airway epithelial integrity in cystic fibrosis. J Cyst Fibros 20, e129-e 139 (2021). https://doi.org:10.1016/j.jcf.2021.09.019
13. Donaldson, S. H. et al. Effect of ivacaftor on mucociliary clearance and clinical outcomes in cystic fibrosis patients with G551D-CFTR. JCI Insight 3 (2018). https://doi.org:10.1172/jci.insight.122695
14. Rock, J. R. et al. Notch-dependent differentiation of adult airway basal stem cells. Cell Stem Cell 8, 639-648 (2011). https://doi.org:10.1016/j.stem.2011.04.003
15. Tsao, P. N. et al. Notch signaling controls the balance of ciliated and secretory cell fates in developing airways. Development 136, 2297-2307 (2009). https://doi.org: 10. 1242/dev.034884
16. Bray, S. J. Notch signalling in context. Nat Rev Mot Cell Biol 17, 722-735 (2016). https://doi.org:10.1038/nrm.2016.94
17. McCaw, T. R. et al. Gamma Secretase Inhibitors in Cancer: A Current Perspective on Clinical Performance. Oncologist 26, e608-e621 (2021). https://doi.org:10.1002/onco.13627
18. Pardo-Saganta, A. et al. Parent stem cells can serve as niches for their daughter cells. Nature 523, 597-601 (2015). https://doi.org:10.1038/nature 14553
19. Zong, D., Ouyang, R., Li, J., Chen, Y. & Chen, P. Notch signaling in lung diseases: focus on Notch 1 and Notch3. Ther Adv Respir Dis 10, 468-484 (2016). https://doi.org:10.1177/1753465816654873
20. Bodas, M. et al. Cigarette Smoke Activates NOTCH3 to Promote Goblet Cell Differentiation in Human Airway Epithelial Cells. Am J Respir Cell Mot Biol 64,426-440(2021). https://doi.org:10.1165/rcmb.2020-03020C
21. Reid, A. T. et al. Blocking Notch3 Signaling Abolishes MUCSAC Production in Airway Epithelial Cells from Individuals with Asthma. Am J Respir Cell Mot Biol 62, 513-523 (2020). https://doi.org:10.1165/rcmb.2019-006900
22. Tindemans, I. et al. Notch signaling licenses allergic airway inflammation by promoting Th2 cell lymph node egress. J Clin Invest 130, 3576-3591 (2020). httpsJ/doi.org:10.1172/JCI128310
23. Stubbs, J. L., Vladar, E. K., Axelrod, J. D. & Kintner, C. Multicilin promotes centriole assembly and ciliogenesis during multiciliate cell differentiation. Nat Cell Biol 14, 140−147 (2012). https://doi.org: 10. 1038/ncb2406
24. Vladar, E. K., Nayak, J. V., Milla, C. E. & Axelrod, J. D. Airway epithelial homeostasis and planar cell polarity signaling depend on multiciliated cell differentiation. JCI Insight 1 (2016). https://doi.org:10.1172/jci.insight.88027
25. Ramirez Williams, L. et al. gamma-Secretase inhibition affects viability, apoptosis, and the stem cell phenotype of endometriotic cells. Acta Obstet Gynecol Scand 98, 1565−1574 (2019). https://doi.org:10.1111/aogs. 13707
26. Allen, F. & Maillard, I. Therapeutic Targeting of Notch Signaling: From Cancer to Inflammatory Disorders. Front Cell Dev Biol 9, 649205 (2021). https://doi.org:10.3389/fcell.2021.649205 027. Panza, F. et al. REVIEW: gamma-Secretase inhibitors for the treatment of Alzheimer's disease: The current state. CNS Neurosci Ther 16, 272-284 (2010). httpsJ/doi.org:10.1111/j. 1755-5949.2010.00164.x
28. Henley, D. B., May, P. C., Dean, R. A. & Siemers, E. R. Development of semagacestat (LY450139), a functional gamma-secretase inhibitor, for the treatment of Alzheimer's disease. Expert Opin Pharmacother 10, 1657−1664 (2009). https://doi.org:10.1517/14656560903044982
29. Braverman, I., Wright, E. D., Wang, C. G., Eidelman, D. & Frenkiel, S. Human nasal ciliary-beat frequency in normal and chronic sinusitis subjects. J Otolaryngol 27, 145−152 (1998).
30. Fulcher, M. L., Gabriel, S., Burns, K. A., Yankaskas, J. R. & Randell, S. H. Well-differentiated human airway epithelial cell cultures. Methods Mol Med 107, 183-206 (2005). https://doi.org:10.1385/1-59259-861-7:183
31. Giuranno, L. et al. NOTCH signaling promotes the survival of irradiated basal airway stem cells. Am J Physiol Lung Cell Mal Physiol 317, L414-L423 (2019). https://doi.org: 10.1152/ajplung.00197.2019
32. Ostrowski, L. E., Hutchins, J. R., Zakel, K. & O'Neal, W. K. Targeting expression of a transgene to the airway surface epithelium using a ciliated cell-specific promoter. Mol Ther 8, 637-645 (2003).
33. Barry, P. J. & Taylor-Cousar, J. L. Triple combination cystic fibrosis transmembrane conductance regulator modulator therapy in the real world-opportunities and challenges. Curr Opin Pulm Med 27, 554-566 (2021). https://doi.org:10.1097/MCP.0000000000000819
34. Plasschaert, L. W. et al. A single-cell atlas of the airway epithelium reveals the CFTR-rich pulmonary ionocyte. Nature 560, 377-381 (2018). https://doi.org: 10. 1038/s41586-018-0394-6
35. Demitrack, E. S. & Samuelson, L. C. Notch regulation of gastrointestinal stem cells. J Physiol 594, 4791-4803 (2016). https://doi.org:10.1113/JP271667
36. Mitani, Y. et al. Differential effects between gamma-secretase inhibitors and modulators on cognitive function in amyloid precursor protein-transgenic and nontransgenic mice. J Neurosci 32, 2037-2050 (2012). https://doi.org: 10. 1523/JNEUROSCI.4264-11.2012
37. Bittner, T. et al. Gamma-secretase inhibition reduces spine density in vivo via an amyloid precursor protein-dependent pathway. J Neurosci 29, 10405−10409 (2009). https://doi.org:10.1523/JNEUROSCI.2288-09.2009
38. Fleisher, A. S. et al. Phase 2 safety trial targeting amyloid beta production with a gamma-secretase inhibitor in Alzheimer disease. Arch Neurol 65, 1031−1038 (2008). https://doi.org: 10. 1001/archneur.65.8.1031
39. Lafkas, D. et al. Therapeutic antibodies reveal Notch control of transdifferentiation in the adult lung. Nature 528, 127−131 (2015). https://doi.org:10.1038/nature 15715
40. Astudillo, L. et al. The Small Molecule IMR-1 Inhibits the Notch Transcriptional Activation Complex to Suppress Tumorigenesis. Cancer Res 76, 3593-3603 (2016). https//doi.org:10.1158/0008-5472.CAN-16-0061
41. Musse, A. A., Meloty-Kapella, L. & Weinmaster, G. Notch ligand endocytosis: mechanistic basis of signaling activity. Semin Cell Dev Biol 23, 429-436 (2012). https://doi.org:10.1016/j.semcdb.2012.01.011

Notwithstanding the appended claims, the disclosure is also defined by the following clauses:

1. A method of treating a respiratory disease characterized by mucus hyper-secretion comprising:
administering a low dose of a GSI to a human patient in need of such treatment; and
wherein the mucus in such patient's lungs is reduced or mucus accumulation in such patient's lungs is inhibited.

2. The method of Clause 1 wherein the respiratory disease is selected from the group consisting of cystic fibrosis, chronic obstructive pulmonary disease, primary ciliary dyskinesis, chronic bronchitis, asthma, idiopathic and secondary bronchiectasis, bronchiolitis obliterans, idiopathic pulmonary fibrosis and other fibrotic lung disorders and respiratory infection, including exacerbations in chronic respiratory disorders and mucus accumulation in response to acute infection.

3. The method of Clause 1 or 2 wherein the GSI is selected from the group consisting of semagacestat, avagacestat, GS-1, DBZ, L-685,458, HMS-906024, crenigakcstat, MRK 560, nirogacestat, RO-4929097, MK-0752, itanapraced, LY-3056480, fosciclopirox, tarenflurbil, and begacestat.

4. The method of Clause 3 wherein said GSI is selected from the group consisting of semagacestat, nirogacestat, MK-0752, RO-492907, or crenigacestat.

5. The method of Clause 4 wherein the GSI is semagacestat.

6. The method of Clause 4 wherein the GSI is MK-0752.

7. The method of Clause 4 wherein the GSI is nirogacestat.

8. The method of Clause 4 wherein the GSI is RO-492907.

9. The method of Clause 4 wherein the GSI is crenigacestat.

10. The method of Clause 2 wherein said administration of GSI is by oral administration.

11. The method of Clause 2 wherein the respiratory disease is cystic fibrosis.

12. The method of Clause 2 wherein the respiratory disease is chronic obstructive pulmonary disease.

13. The method of Clause 5 wherein semagacestat is administered orally in an amount of from about 0.1 mg to about 50 mg daily.

14. The method of Clause 13 wherein about 0.5 mg to about 40 mg of semagacestat is administered daily.

15. The method of Clause 14 wherein about 0.5 mg to about 30 mg of semagacestat is administered daily.

16. The method of Clause 15 wherein about 0.5 mg to about 20 mg of semagacestat is administered daily.

17. The method of Clause 7 wherein the nirogacestat is administered orally in an amount of from about 8 ug to about 0.9 mg daily.

18. The method of Clause 17 wherein about 10 ug to about 300 ug of nirogacestat is administered daily.

19. The method of Clause 8 wherein the RO-492907 is administered orally in an amount of from about 0.1 mg to about 20 mg daily.

20. The method of Clause 19 wherein about 0.1 mg to about 10 mg of RO-492907 is administered daily.

21. The method of Clause 20 wherein about 0.1 mg to about 5 mg of RO-492907 is administered daily.

22. The method of Clause 6 wherein MK-0752 is administered orally in an amount of from about 0.1 mg to about 40 mg daily.

23. The method of Clause 22 wherein about 0.1 mg to about 20 mg of MK-0752 is administered daily.

24. The method of Clause 23 wherein about 0.1 mg to about 10 mg of MK-0752 is administered daily.

25. The method of any of Clauses 1-24, wherein the low dose of the GSI does not cause an adverse gastrointestinal event.

26. The method of Clause 25, wherein the adverse gastrointestinal event is selected from the group consisting of decreased appetite, nausea, vomiting, weight loss, diarrhea, and gastrointestinal bleeding.

27. A method of treating a respiratory disease characterized by mucus hyper-secretion comprising:
systemically administering to a human patient in need of such treatment a therapeutically effective amount of semagacestat, wherein said patient's semagacestat plasma concentration at steady state following about 1 week or after about 2 weeks or more of administering the therapeutically effective amount has an AUC less than 1220 ng·hr/mL, and
   wherein the administration of semagacestat is effective in reducing mucus in such patient's lungs or inhibiting mucus accumulation in such patient's lungs.

28. The method of Clause 27 wherein the respiratory disease is selected from the group consisting of cystic fibrosis, chronic obstructive pulmonary disease, primary ciliary dyskinesis, chronic bronchitis, asthma, idiopathic and secondary bronchiectasis, bronchiolitis obliterans, idiopathic pulmonary fibrosis and other fibrotic lung disorders and respiratory infection, including exacerbations in chronic respiratory disorders and mucus accumulation in response to acute infection.

29. The method of Clause 28 wherein said patient's semagacestat plasma concentration at steady state following multiple dose administration comprises an AUC less than 600 ng4 hr/mL

30. The method of Clause 29 wherein said patient's semagacestat plasma concentration at steady state following multiple dose administration comprises an AUC less than 250 ng4 hr/mL

31. The method of Clause 30 wherein the respiratory disease is cystic fibrosis.

32. The method of Clause 31 wherein the respiratory disease is chronic obstructive pulmonary disease.

33. The method of Clause 27-32, wherein the therapeutically effective amount of semagacestat does not cause an adverse gastrointestinal event.

34. The method of Clause 33, wherein the adverse gastrointestinal event is selected from the consisting of decreased appetite, nausea, vomiting, weight loss, diarrhea, and gastrointestinal bleeding.

35. A method of treating cystic fibrosis comprising:
administering an effective amount of a GSI to a human patient being administered or need administration of one or more CFTR modulators,
and wherein the mucus in such patient's lungs is reduced or mucus accumulation in such patient's lungs is inhibited upon administration of the GSI.

36. The method of Clause 35 wherein the GSI is selected from the group consisting of semagacestat, avagacestat, GS-1, DBZ, L-685,458, BMS-906024, crenigasccstat, MRK 560, nirogacestat, RO-4929097, MK-0752, itanapraced. LY-3056480, fosciclopirox, tarenflurbil, and begacestat.

37. The method of Clause 36 wherein the GSI is selected from the group consisting of semagacestat., nirogacestat, MK-0752, RO-492907, or crenigacestat.

38. The method of Clause 37 wherein the GSI is semagacestat.

39. The method of Clause 37 wherein the GSI is MK-0752.

40. The method of Clause 37 wherein the GSI is nirogacestat.

41. The method of Clause 37 wherein the GSI is RO-492907.

42. The method of Clause 37 wherein the GSI is ccenigacestat.

43. The method of Clause 35 wherein said administration of GSI is by oral administration.

44. The method of Clause 35 wherein the CFTR modulator is a CFTR potentiator.

45. The method of Clause 35 wherein the CFTR modulator is a CFTR corrector.

46. The method of Clause 35 wherein the CFTR modulator is a CFTR amplifier.

47. The method of Clause 35 wherein the CFTR modulator is selected from the group consisting of ivacaftor, lumacaftor, tezacaftor, elexacaftor and combinations thereof.

48. The method of Clause 35 wherein the GSI is administered daily.

49. The method of Clause 48 wherein the GSI is administered for up to thirty days and then stopped.

50. The method of Clause 35 wherein the GSI is administered weekly.

51 The method of any of Clauses 35-50, wherein the GSI is administered systemically.

52. The method of Clause 51, wherein the systemic administration is by oral administration

53. The method of any of Clauses 35-50, wherein the GSI is administered locally.

54. The method of Clause 53, wherein the local administration is by inhalation administration.

55. The method of any of Clauses 35-54, wherein the effective amount of the GSI is a low dose of the GSI.

56. The method of any of Clause 35-55, wherein the effective amount of the GSI does not cause an adverse gastrointestinal event.

57. The method of Clause 56, wherein the adverse gastrointestinal event is selected from the group consisting of decreased appetite, nausea, vomiting, weight loss, diarrhea, and gastrointestinal bleeding.

58. A method of treating cystic fibrosis comprising:
administering an effective amount of a GSI to a human patient taking a CFTR modulator;
wherein the GSI is selected from the group consisting of semagacestat, avagacestat, GS-1, DBZ. L-685,458, BMS-906024, crenigascestat, MRK 560, nirogacestat, RO-4929097, MK-0752, itanapraced, LY-3056480, fosciclopirox, tarenflurbil, and begacestat;
wherein the CFTR modulator is selected from the group consisting of ivacaftor, lumacaftor, tezacaftor, elexacaftor and combinations thereof; and
wherein the mucus in such patient's lungs is reduced or mucus accumulation in such patient's lungs is inhibited upon administration of the GSI.

59. The method of Clause 58 wherein the GSI is selected from the group consisting of semagacestat., nirogacestat, MK-0752, RO-492907, or crenigacestat.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of treating a respiratory disease characterized by mucus hyper-secretion comprising:
administering a low dose of a GSI to a human patient in need of such treatment; and
wherein the mucus in such patient's lungs is reduced or mucus accumulation in such patient's lungs is substantially ameliorated or prevented upon administration of the GSI.

2. The method of claim 1, wherein the low dose is an effective amount to treat the respiratory disease characterized by mucus hyper-secretion and is a lower dose as compared to a dose of the GSI suitable for administering to a patient suffering from a neurodegenerative disorder, an oncology disorder, or a respiratory disease not characterized by mucus hyper-secretion.

3. The method of claims 1 or 2 wherein the low dose of a GSI yields a peak plasma level in the submicromolar range.

4. The method of any of claims 1-3, wherein the low dose of the GSI does not cause an adverse gastrointestinal event in the human patient.

5. The method of any of claims 1-4, wherein the adverse gastrointestinal event is selected from the group consisting of decreased appetite, nausea, vomiting, weight loss, diarrhea, and gastrointestinal bleeding.

6. The method of any of claims 1-5, wherein the respiratory disease is selected from the group consisting of cystic fibrosis, chronic obstructive pulmonary disease, primary ciliary dyskinesis, chronic bronchitis, asthma, idiopathic and secondary bronchiectasis, bronchiolitis obliterans, idiopathic pulmonary fibrosis and other fibrotic lung disorders and respiratory infection, including exacerbations in chronic respiratory disorders and mucus accumulation in response to acute infection.

7. The method of any of the preceding claims wherein the GSI is selected from the group consisting of semagacestat, avagacestat, GS-1, DBZ, L-685,458, BMS-906024, crenigascestat, MRK 560, nirogacestat, RO-4929097, MK-0752, itanapraced, LY-3056480, fosciclopirox, tarenflurbil, and begacestat.

8. The method of claim 7, wherein said GSI is selected from the group consisting of semagacestat, nirogacestat, MK-0752, RO-492907, or crenigacestat.

9. The method of claim 8, wherein the GSI is semagacestat.

10. The method of claim 8, wherein the GSI is MK-0752.

11. The method of claim 8, wherein the GSI is nirogacestat.

12. The method of claim 8, wherein the GSI is RO-492907.

13. The method of claim 8, wherein the GSI is crenigacestat.

14. The method of any of the preceding claims wherein said administration of GSI is by oral administration.

15. The method of any of the preceding claims wherein the respiratory disease is cystic fibrosis or chronic obstructive pulmonary disease.

16. A method of treating a respiratory disease characterized by mucus hyper-secretion comprising:
systemically administering to a human patient in need of such treatment a therapeutically effective amount of semagacestat, wherein said patient's semagacestat plasma concentration at steady state following multiple dose administration comprises an AUC less than 1220 ngshr/mL, and
wherein the administration of semagacestat is effective in reducing mucus in such patient's lungs or inhibiting mucus accumulation in such patient's lungs.

17. The method of claim 16 wherein the respiratory disease is selected from the group consisting of cystic fibrosis, chronic obstructive pulmonary disease, primary ciliary dyskinesis, chronic bronchitis, asthma, idiopathic and secondary bronchiectasis, bronchiolitis obliterans, idiopathic pulmonary fibrosis and other fibrotic lung disorders and respiratory infection, including exacerbations in chronic respiratory disorders and mucus accumulation in response to acute infection.

18. The method of claim 17 wherein the respiratory disease is cystic fibrosis or chronic obstructive pulmonary disease.

19. The method of any of claims 16-18, wherein the semagacestat is administered in an amount of from about 0.1 mg to about 50 mg daily.

20. The method of claim 19, wherein about 0.5 mg to about 40 mg of semagacestat is administered daily.

21. The method of claim 20, wherein about 0.5 mg to about 30 mg of semagacestat is administered daily.

22. The method of claim 21, wherein about 0.5 mg to about 20 mg of semagacestat is administered daily.

23. The method of any of claims 16-22, wherein the therapeutically effective amount of semagacestat does not cause an adverse gastrointestinal event.

24. The method of claim 23, wherein the adverse gastrointestinal event is selected from the group consisting of decreased appetite, nausea, vomiting, weight loss, diarrhea, and gastrointestinal bleeding.

25. A method of treating cystic fibrosis comprising:
administering an effective amount of a GSI to a human patient being administered or in need of a CFTR modulator,
wherein the mucus in such patient's lungs is reduced or mucus accumulation in such patient's lungs is inhibited.

26. The method of claim 25 wherein the GSI is selected from the group consisting of semagacestat, avagacestat, GS-1, DBZ, L-685.458, BMS-906024, crenigascestat, MRK 560, nirogacestat, RO-4929097, MK-0752, itanapraced, LY-3056480, fosciclopirox, tarenflurbil, and begacestat.

27. The method of claim 26 wherein the GSI is selected from the group consisting of semagacestat, nirogacestat, MK-0752, RO-492907, or crenigacestat.

28. The method of any of claims 25-27, wherein the GSI is administered systemically.

29. The method of claim 28, wherein the systemic administration is by oral administration.

30. The method of any of claims 25-27, wherein the GSI is administered locally.

31. The method of claim 30, wherein the local administration is by inhalation administration.

32. The method of any of claims 25-31, wherein the effective amount of the GSI is a low dose of the GSI.

33. The method of any of claims 25-32, wherein the effective amount of the GSI does not cause an adverse gastrointestinal event.

34. The method of claim 33, wherein the adverse gastrointestinal event is selected from the group consisting of decreased appetite, nausea, vomiting, weight loss, diarrhea, and gastrointestinal bleeding.
